(19) United States
(12) Patent Application Publication
(10) Pub. No.: US 2009/0230855 A1
(43) Pub. Date: Sep. 17, 2009

(54) Novel organic compound and organic light emitting device comprising the same

(76) Inventors: Myeong-suk Kim, Hwaseong-si (KR); Dong-woo Shin, Seoul (KR); Byoung-ki Choi, Hwaseong-si (KR); Yu-jin Kim, Suwon-si (KR); Tae-yong Noh, Seoul (KR); O-hyun Kwon, Seoul (KR); Tae-woo Lee, Seoul (KR)

Correspondence Address: ROBERT E. BUSHNELL & LAW FIRM, 2029 K STREET NW, SUITE 600, WASHINGTON, DC, 20006-1004, US

(21) Appl. No.: 12/382,356

(22) Filed: Mar. 13, 2009

(30) Foreign Application Priority Data
Mar. 14, 2008 (KR) 10-2008-0024059

(51) Int. Cl. H01J 1/63; C07D 333/52

(52) U.S. Cl. 313/504; 549/49

(57) ABSTRACT

Provided are a compound represented by Formula 1 and an organic light emitting device including the same:

X—Ar₁—Ar₂—Y (1)

where Ar₁, Ar₂, X, and Y are the same as in the detailed description. The organic light emitting device including the compound has low driving voltage, high brightness, high efficiency, and high color purity.

### CROSS-REFERENCE TO RELATED PATENT APPLICATIONS AND CLAIM OF PRIORITY

This application claims the benefit of Korean Patent Application No. 10-2008-0024059, filed on Mar. 14, 2008, in the Korean Intellectual Property Office, the disclosure of which is incorporated herein in its entirety by reference.

### BACKGROUND OF THE INVENTION

1. Field of the Invention

The present invention relates to a compound and an organic light emitting device (OLED) including the same, and more particularly, to a compound for an organic layer of an organic light emitting device, and an organic light emitting device including the organic layer containing the compound.

2. Description of the Related Art

Organic light emitting devices (OLED) are self-emission display devices, and generate light by recombination of electrons and holes when a current is provided to a fluorescent or phosphorescent organic compound thin film (hereinafter, referred to as an organic layer). OLEDs can be manufactured to be lightweight, include a relatively small number of components and thus have a simple manufacturing process, can produce high-quality images, and have wide viewing angles. In addition, OLEDs can completely produce moving images with high color purity and have low power consumption and low driving voltage. Due to these advantages, OLED can be used in portable applications.

Eastman Kodak Co. developed a multi-layer OLED including an aluminum quinolinol complex layer and a triphenylamine derivative layer (see U.S. Pat. No. 4,885,211), and a light emission wavelength range was widened to be in a range from an ultra-violet (UV) wavelength to an infrared wavelength by using a small molecular material to form an organic emission layer (see U.S. Pat. No. 5,151,629.)

Light emitting devices are self-emission devices, and have wide viewing angles, excellent contrast properties, and quick response speeds. Light emitting devices can be categorized into inorganic light emitting devices including an emission layer formed of an inorganic compound and OLEDs including an emission layer formed of an organic compound. Compared to inorganic light emitting devices, OLEDs have excellent brightness, low driving voltages, and quick response speeds, and can produce full-color images. Therefore, research into OLEDs is being actively performed.

In general, an OLED has a structure of anode/organic emission layer/cathode. An OLED can also have other structures, such as a structure of anode/hole injection layer/hole transport layer/emission layer/electron transport layer/electron injection layer/cathode, or a structure of anode/hole injection layer/hole transport layer/emission layer/hole blocking layer/electron transport layer/electron injection layer/cathode.

Materials used in organic light emitting devices can be categorized into vacuum deposition material and solution coating material according to a method of forming an organic layer. The vacuum deposition material can be any material that can produce a vapor pressure of 10⁻⁶ torr or more at 500° C. or lower. For example, the vacuum deposition material may be a small molecular material having a molecular weight of 1200 or less. The solution coating material can be any material that can retain high solubility with respect to a solvent. For example, the solution coating material can be aromatic or heterocyclic.

JP Patent Publication No. 1999-003782 discloses an anthracene substituted with two naphthyl groups, which can be used to form an emission layer or a hole injection layer. However, OLEDs including this compound do not have sufficient driving voltage, brightness, efficiency, and color purity properties. Thus, there is a need to develop an OLED having desirable low driving voltage, high brightness, high efficiency, and high color purity properties.

### SUMMARY OF THE INVENTION

The present invention provides a compound capable of improving driving voltage properties, efficiency, and color purity when used in an organic light emitting device (OLED) and an organic light emitting device including the compound.

According to an aspect of the present invention, there is provided a compound represented by Formula 1:

X—Ar₁—Ar₂—Y <Formula 1>

where Ar₁ is a substituted or unsubstituted C₅-C₅₀ arylene group, a substituted or unsubstituted C₅-C₃₀ heteroarylene group, a substituted or unsubstituted C₅-C₅₀ alkenylene group, or a combination thereof by a single bond;

Ar₂ is a substituted or unsubstituted C₅-C₁₅ arylene group, a substituted or unsubstituted C₅-c₁₅ heteroarylene group, a substituted or unsubstituted C₅-C₁₅ alkenylene group, a substituted or unsubstituted C₅-C₁₅ cycloalkylene group, or a combination thereof by a single bond;

X is each independently hydrogen, halogen, a cyano group, a hydroxyl group, a substituted or unsubstituted C₁-C₂₀ alkyl group, a substituted or unsubstituted C₃-C₂₀ cycloalkyl group, a substituted or unsubstituted C₅-C₂₀ heterocycloalkyl group, a substituted or unsubstituted C₁-C₂₀ alkoxy group, a substituted or unsubstituted C₆-C₃₀ aryl group, a substituted or unsubstituted C₆-C₃₀ arylalkyl group, a substituted or unsubstituted C₂-C₃₀ heteroaryl group, a substituted or unsubstituted C₃-C₃₀ aryl amino group, a substituted or unsubstituted C₃-C₃₀ arylsilane group, a group represented by Formula 2a or a group represented by Formula 2b; and

Y is a group represented by Formula 2a or Formula 2b:

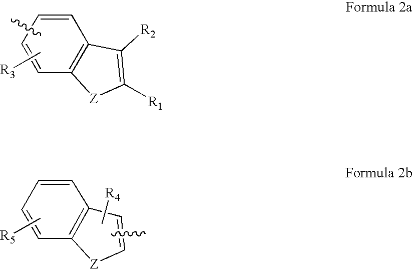

where Z is S, O, N, Se, or SO₂;

R₁ through R₅ are each independently hydrogen, halogen, a cyano group, a hydroxyl group, a substituted or unsubstituted C₁-C₂₀ alkyl group, a substituted or unsubstituted C₃-C₂₀ cycloalkyl group, a substituted or unsubstituted C₅-C₂₀ heterocycloalkyl group, a substituted or unsubstituted C₁-C₂₀ alkoxy group, a substituted or unsubstituted C₆-C₃₀ aryl group, a substituted or unsubstituted C₆-C₃₀ arylalkyl group, a substituted or unsubstituted C₂-C₃₀ heteroaryl group, a substituted or unsubstituted C₃-C₃₀ aryl amino group, or a substituted or unsubstituted C₃-C₃₀ arylsilane group.

According to another aspect of the present invention, there is provided an organic light emitting device comprising: a first electrode; a second electrode; and at least one organic layer interposed between the first electrode and the second electrode, wherein the at least one organic layer comprises the compound according to the present invention as described above.

### BRIEF DESCRIPTION OF THE DRAWINGS

A more complete appreciation of the invention, and many of the attendant advantages thereof, will be readily apparent as the same becomes better understood by reference to the following detailed description when considered in conjunction with the accompanying drawings in which like reference symbols indicate the same or similar components, wherein:

FIGS. 1A through 1C illustrate schematic stacked structures of organic light emitting devices according to embodiments of the present invention; and

FIG. 2 illustrates ultra-violet (UV) and photoluminescence (PL) spectra of a compound according to another embodiment of the present invention.

### DETAILED DESCRIPTION OF THE INVENTION

The present invention will now be described more fully with reference to the accompanying drawings, in which exemplary embodiments of the invention are shown.

A compound according to an embodiment of the present invention is represented by Formula 1:

X—Ar₁—Ar₂—Y <Formula 1>

where Ar₁ is a substituted or unsubstituted C₅-C₅₀ arylene group, a substituted or unsubstituted C₅-C₃₀ heteroarylene group, a substituted or unsubstituted C₅-C₅₀ alkenylene group, or a combination thereof connected via a single bond;

Ar₂ is a substituted or unsubstituted C₅-C₁₅ arylene group, a substituted or unsubstituted C₅-C₁₅ heteroarylene group, a substituted or unsubstituted C₅-C₁₅ alkenylene group, a substituted or unsubstituted C₅-C₁₅ cycloalkylene group, or a combination thereof by a single bond;

X is each independently hydrogen, halogen, a cyano group, a hydroxyl group, 11 a substituted or unsubstituted C₁-C₂₀ alkyl group, a substituted or unsubstituted C₃-C₂₀ cycloalkyl group, a substituted or unsubstituted C₅-c₂₀ heterocycloalkyl group, a substituted or unsubstituted C₁-C₂₀ alkoxy group, a substituted or unsubstituted C₆-C₃₀ aryl group, a substituted or unsubstituted C₆-C₃₀ arylalkyl group, a substituted or unsubstituted C₂-C₃₀ heteroaryl group, a substituted or unsubstituted C₃-C₃₀ aryl amino group, a substituted or unsubstituted C₃-C₃₀ arylsilane group, a group represented by Formula 2a or a group represented by Formula 2b; and

Y is a group represented by Formula 2a or Formula 2b:

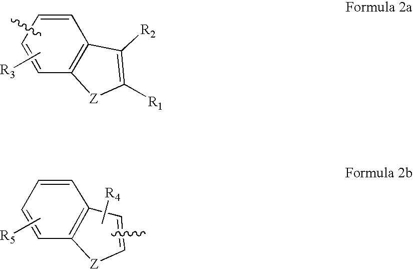

where Z is S, O, N, Se, or SO₂;

R₁ through R₅ are each independently hydrogen, halogen, a cyano group, a hydroxyl group, a substituted or unsubstituted C₁-C₂₀ alkyl group, a substituted or unsubstituted C₃-C₂₀ cycloalkyl group, a substituted or unsubstituted C₅-C₂₀ heterocycloalkyl group, a substituted or unsubstituted C₁-C₂₀ alkoxy group, a substituted or unsubstituted C₆-C₃₀ aryl group, a substituted or unsubstituted C₆-C₃₀ arylalkyl group, a substituted or unsubstituted C₂-C₃₀ heteroaryl group, a substituted or unsubstituted C₃-C₃₀ aryl amino group, or a substituted or unsubstituted C₃-C₃₀ arylsilane group.

In Formulae 2a and 2b, “ [symbol] ” denotes a linking group of Formula 1. The group represented by Formula 2a or 2b may have the following structures:

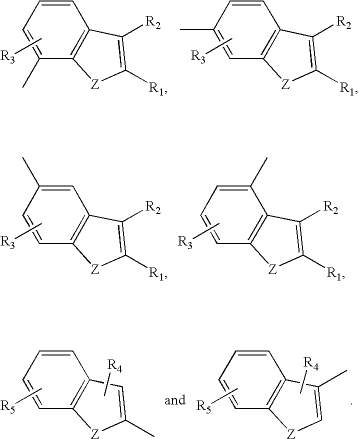

An exemplary unsubstituted alkyl group used in formulae of the present invention may be methyl, ethyl, propyl, isobutyl, sec-butyl, tert-butyl, pentyl, iso-amyl, or hexyl. In a substituted alkyl group used in formulae of the present invention, at least one hydrogen atom of the unsubstituted alkyl group is substituted with a substituent.

An exemplary unsubstituted cycloalkyl group used in formulae of the present invention may be a cyclohexyl group or a cyclopentyl group. In a substituted cycloalkyl group used in formulae of the present invention, at least one hydrogen atom of the unsubstituted cycloalkyl group is substituted with a substituent.

An exemplary unsubstituted alkoxy group used in formulae of the present invention may be methoxy, ethoxy, phenyloxy, cyclohexyloxy, naphthyloxy, isopropyloxy, or diphenyloxy. In a substituted alkoxy group used in formulae of the present invention, at least one hydrogen atom of the unsubstituted alkoxy group is substituted with a substituent.

An aryl group used in formulae of the present invention is a carbocyclic aromatic system having at least one aromatic group, and, when two or more aromatic groups are included, at least one ring are pendent or fused with another aromatic group. Examples of an unsubstituted aryl group include a phenyl group, an ethylphenyl group, an ethylbiphenyl group, an o-, m- and p-fluorophenyl group, a dichlorophenyl group, a dicyanophenyl group, a trifluoromethoxyphenyl group, an o-, m-, and p-tolyl group, an o-, m- and p-cumenyl group, a mesityl group, a phenoxyphenyl group, a (α, α-dimethylbenzene)phenyl group, a (N,N′-dimethyl)aminophenyl group, a pentalenyl group, an indenyl group, a naphthyl group, a methylnaphthyl group, an anthracenyl group, an azurenyl group, a heptalenyl group, an acenaphthylenyl group, a phenalenyl group, a fluorenyl group, an anthraquinolyl group, a methylanthryl group, a phenanthryl group, a triphenylene group, a pyrenyl group, a chrysenyl group, an ethyl-chrysenyl group, a picenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a pyranthrenyl group, an ovalenyl group, and a carbazolyl group. At least one hydrogen atom of the unsubstituted aryl group may be substituted with a substituent.

An unsubstituted arylalkyl group used in formulae of the present invention may be obtained by substituting one or more hydrogen atoms of the aryl group described above with alkyl, for example, low alkyl such as methyl, ethyl, or propyl. Examples of the unsubstituted arylalkyl group include benzyl and phenylethyl. At least one hydrogen atom of the unsubstituted arylalkyl group may be substituted with a substituent.

An unsubstituted heteroaryl group used in formulae of the present invention is a carbocyclic aromatic system having at least one aromatic group containing non-carbon ring atoms, for example, N, O, P and S. In the substituted heteroaryl group, at least one hydrogen atom of the unsubstituted heteroaryl group is substituted with a substituent. Examples of the unsubstituted heteroaryl group include a pyrazolyl group, an imidazolyl group, an oxazolyl group, a thiazolyl group, a triazolyl group, a tetrazolyl group, an oxadiazolyl group, a pyridinyl group, a pyridazinyl group, a pyrimidinyl group, a triazinyl group, a carbazolyl group, and an indolyl group.

Examples of an unsubstituted arylene group used in formulae of the present invention include phenylene and biphenylene. In a substituted arylene group used in formulae of the present invention, at least one hydrogen atom of the unsubstituted arylene group is substituted with a substituent.

An unsubstituted heteroarylene group used in formulae of the present invention is a C6-C30 monocyclic or bicyclic aromatic organic compound where one, two, or three hetero atoms are selected from N, O, P and S and the other ring atoms are carbons. In a substituted heteroarylene group used in formulae of the present invention, at least one hydrogen atom of the unsubstituted heteroarylene group is substituted with a substituent.

An unsubstituted aryl amino group used in formulae of the present invention may be represented by —Ar—N(Q1)(Q2) where Q1 and Q2 are each independently hydrogen, a substituted or unsubstituted C₁-C₂₀ alkyl group, a substituted or unsubstituted C₆-C₃₀ aryl group, a substituted or unsubstituted C₂-C₃₀ heteroaryl group, a substituted or unsubstituted C₅-C₂₀ cycloalkyl group or a substituted or unsubstituted C₅-C₃₀ heterocycloalkyl group. Examples of the unsubstituted aryl amino group include a diphenylamino group.

An unsubstituted arylsilane group used in formulae of the present invention is represented by —Ar—Si(Q3)(Q4)(Q5) where Q3, Q4 and Q5 are each independently hydrogen, a substituted or unsubstituted C₁-C₂₀ alkyl group, a substituted or unsubstituted C₆-C₃₀ aryl group, a substituted or unsubstituted C₂-C₃₀ heteroaryl group, a substituted or unsubstituted C₅-C₂₀ cycloalkyl group or a substituted or unsubstituted C₅-C₃₀ heterocycloalkyl group.

The term “substituted” used to define substituents used in formulae according to the present invention refers to substituting one or more hydrogen atoms with any substituent. The substituent may include at least one substituent selected from the group consisting of —F; —Cl; —Br; —CN; —NO₂; —OH; a C₁-C₅₀ alkyl group that is unsubstituted or substituted with —F, —Cl, —Br, —CN, —NO₂ or —OH; a C₁-C₅₀ alkoxy group that is unsubstituted or substituted with —F, —Cl, —Br, —CN, —NO₂ or —OH; a C₆-C₅₀ aryl group that is unsubstituted or substituted with a C₁-C₅₀ alkyl group, a C₁-C₅₀ alkoxy group, —F, —Cl, —Br, —CN, —NO₂ or —OH; a C₂-C₅₀ heteroaryl group that is unsubstituted or substituted with a C₁-C₅₀ alkyl group, a C₁-C₅₀ alkoxy group, —F, —Cl, —Br, —CN, —NO₂ or —OH; a C₅-C₅₀ cycloalkyl group that is unsubstituted or substituted with a C₁-C₅₀ alkyl group, a C₁-C₅₀ alkoxy group, —F, —Cl, —Br, —CN, —NO₂ or -OH; a C₅-C₅₀ heterocycloalkyl group that is unsubstituted or substituted with a C₁-C₅₀ alkyl group, a C₁-C₅₀ alkoxy group, —F, —Cl, —Br, —CN, —NO₂ or —OH; or a group represented by -N(Q6)(Q7) where Q6 and Q7 are each independently hydrogen, a C₁-C₅₀ alkyl group, or a C₆-C₅₀ aryl group substituted with a C₁-C₅₀ alkyl group.

In the present specification, the term “derivative” refers to a group obtained by substituting at least one hydrogen atom of the groups described above with the substituents described above.

Specifically, the desirable substituent may be methoxy, a phenyl group, a tolyl group, a naphthyl group, a pyrenyl group, a phenanthrenyl group, a fluorenyl group, an imidazolinyl group, an indolyl group, a quinolinyl group, a diphenylamino group, a 2,3-di-p-toylaminophenyl group or a triphenylsilyl group.

In Formula 1, Ar₁ may be selected from groups having the following structures:

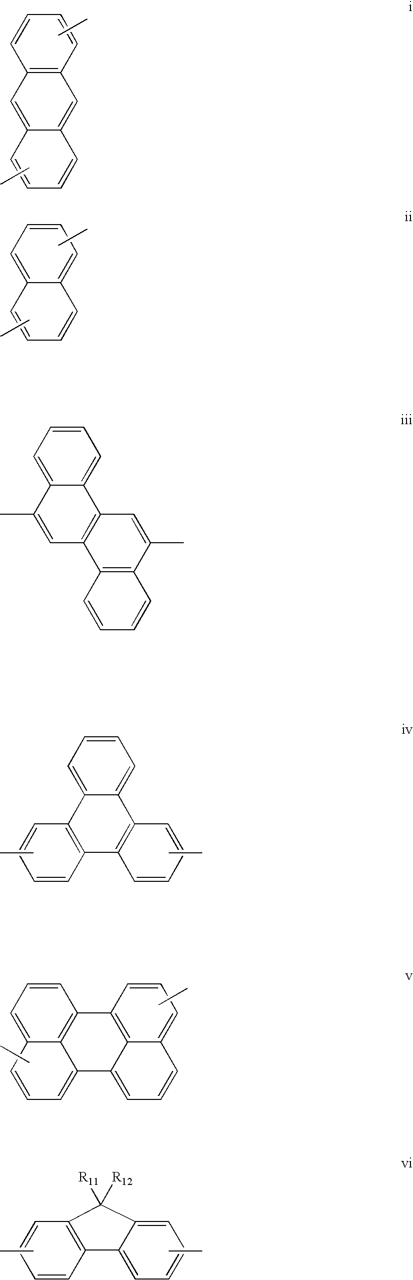

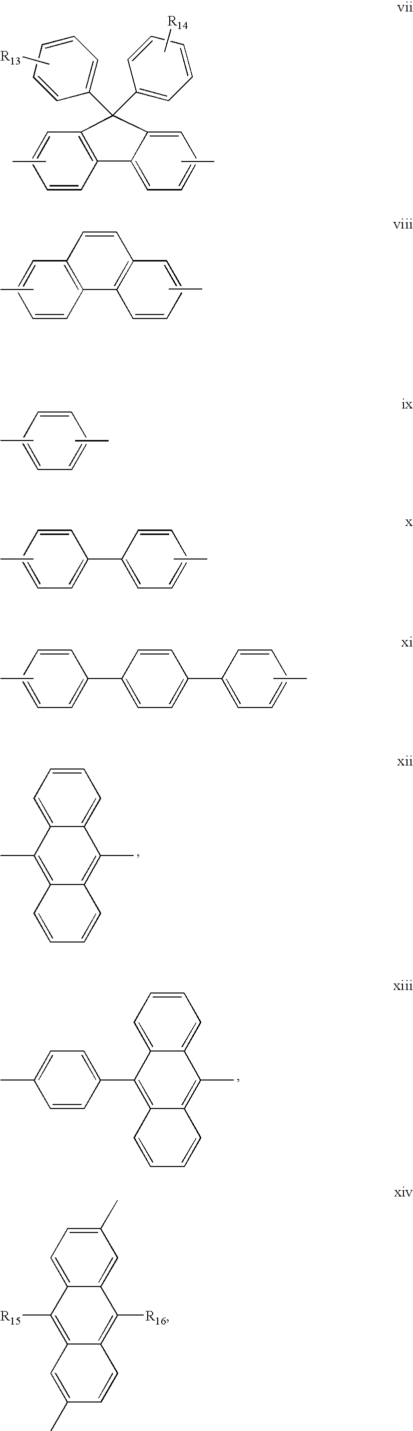

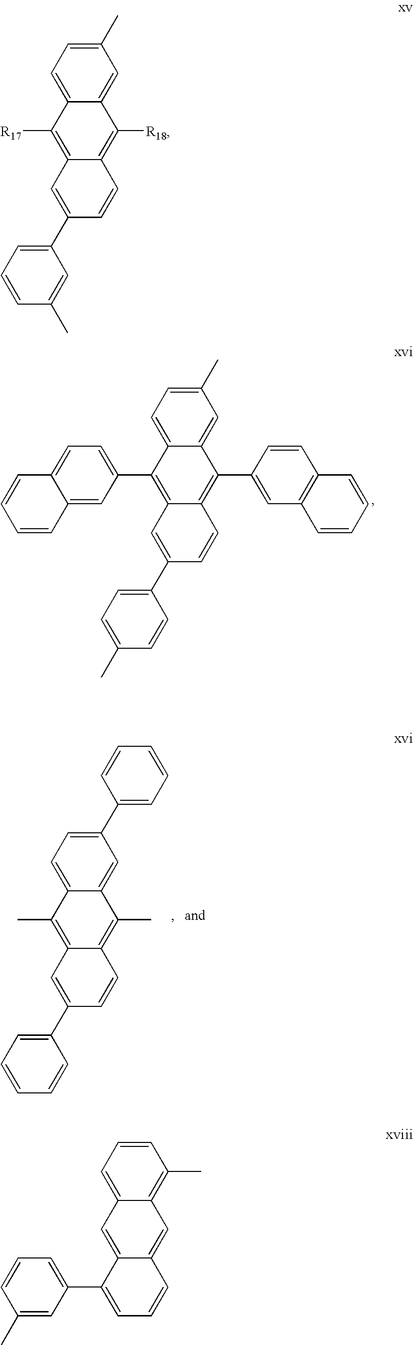

where R₁₁, R₁₂, R₁₃, R₁₄, R₁₅, R₁₆, R₁₇ and R₁₈ are each independently hydrogen, a substituted or unsubstituted C₁-C₂₀ alkyl group, a substituted or unsubstituted C₆-C₃₀ aryl group, a substituted or unsubstituted C₂-C₃₀ heteroaryl group, a substituted or unsubstituted C₅-C₂₀ cycloalkyl group or a substituted or unsubstituted C₅-C₃₀ heterocycloalkyl group.

Ar₁ determines a major emission region of a compound. In addition, due to inclusion of Ar₁, the compound according to the embodiment of the present invention is thermally stable.

In Formula 1, Ar₂ may have any one of the following structures:

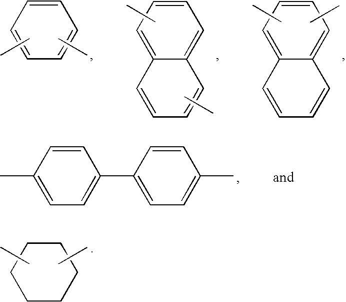

Due to inclusion of Ar₂, a compound according to the embodiment of the present invention is more stabilized, and due to bended bonding, the compound according to the embodiment of the present invention generates light having a short wavelength.

Due to inclusion of the compounds represented by Formulae 2a and 2b, the compound represented by Formula 1 has high thermal stability and high photochemical stability.

R₁ through R₅, and X are each independently hydrogen, a C₁-C₂₀ alkyl group, a C₁-C₂₀ alkoxy group, a phenyl group, a biphenyl group, a pentalenyl group, an indenyl group, a naphthyl group, a biphenylenyl group, an anthracenyl group, an azurenyl group, a heptalenyl group, an acenaphthylenyl group, a phenalenyl group, a fluorenyl group, a methylanthryl group, a phenanthrenyl group, a triphenylenyl group, a pyrenyl group, a chrysenyl group, an ethyl-chrysenyl group, a picenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a fluorenyl group, a pyranthrenyl group, an ovalenyl group, a carbazolyl group, a thiophenyl group, an indolyl group, a purinyl group, a benzimidazolyl group, a quinolinyl group, a benzothiophenyl group, a parathiazinyl group, a pyrrolyl group, a pyrazolyl group, an imidazolyl group, an imidazolinyl group, an oxazolyl group, a thiazolyl group, a triazolyl group, a tetrazolyl group, an oxadiazolyl group, a pyridinyl group, a pyridazinyl group, a pyrimidinyl group, a pyrazinyl group, a thianthrenyl group, a cyclopentyl group, a cyclohexyl group, an oxiranyl group, a pyrrolidinyl group, a pyrazolidinyl group, an imidazolyl group, a piperidinyl group, a piperazinyl group, a morpholinyl group, a di(C₆-C₅₀ aryl)amino group, a tri(C₆-C₅₀aryl)silyl group, or derivatives thereof.

X can also be selected from the groups represented by Formula 2a or Formula 2b.

Due to inclusion of R₁ through R₅ and X, the compound represented by Formula 1 has high solubility and good amorphous properties, and thus has high film processibility.

The compound represented by Formula 1 may be a compound represented by any one of Formulae 3 through 23:

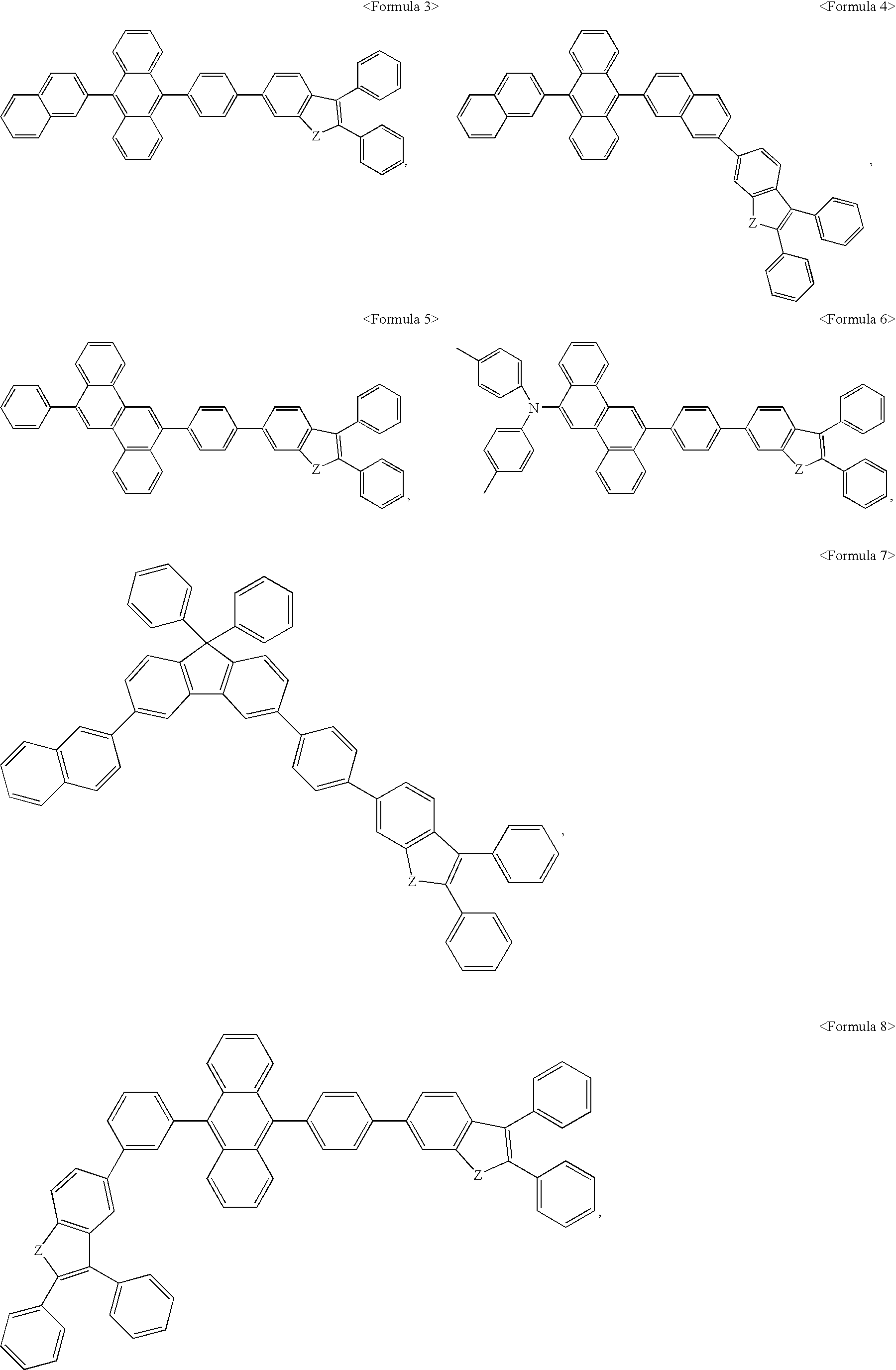

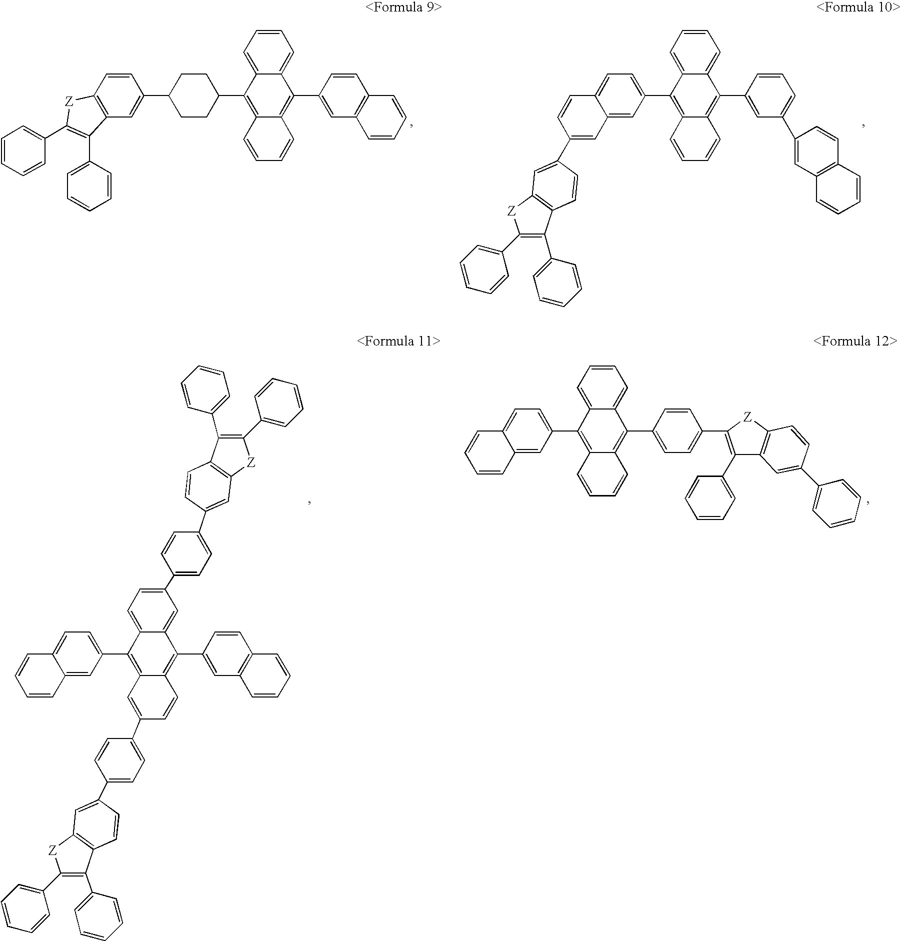

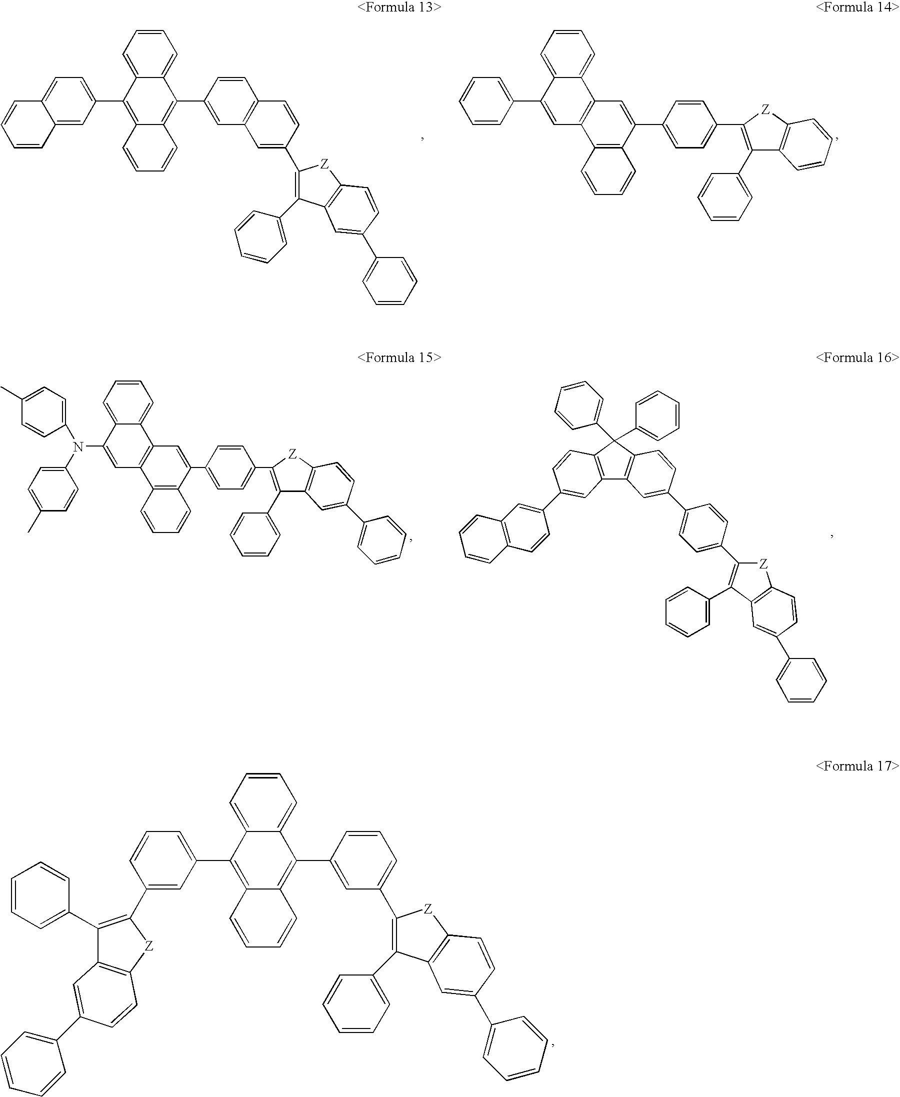

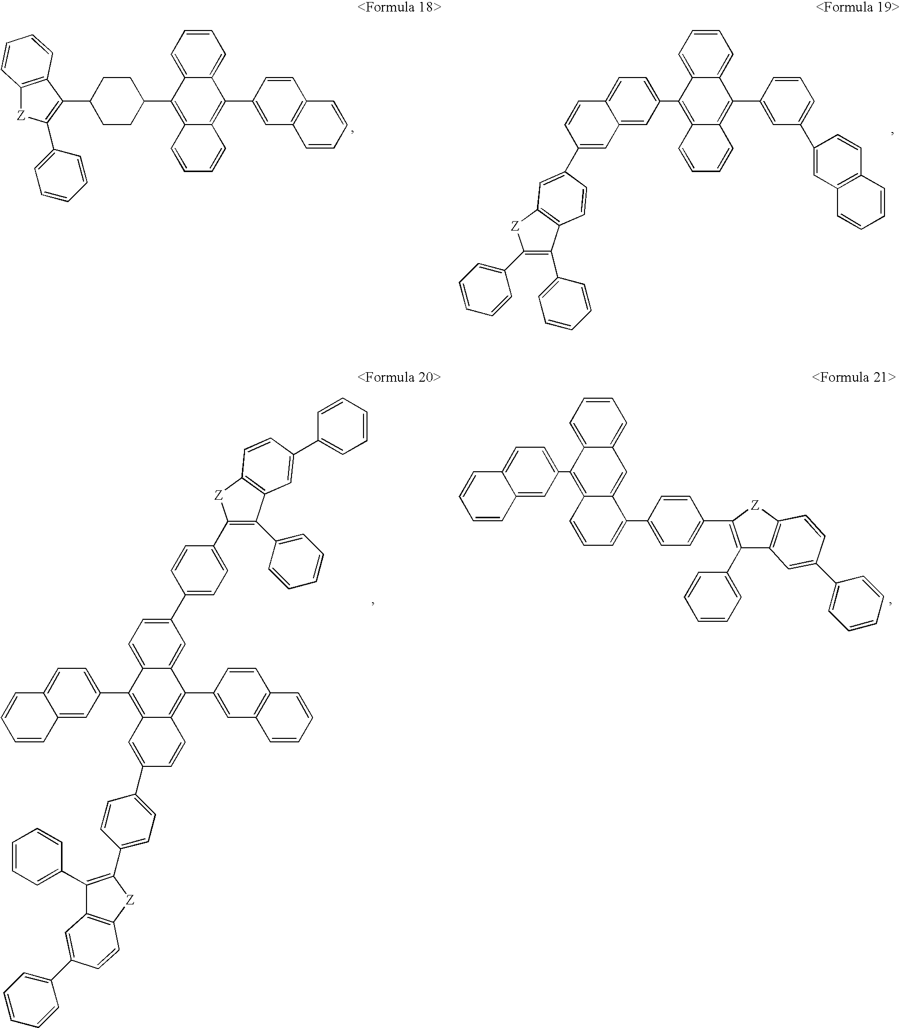

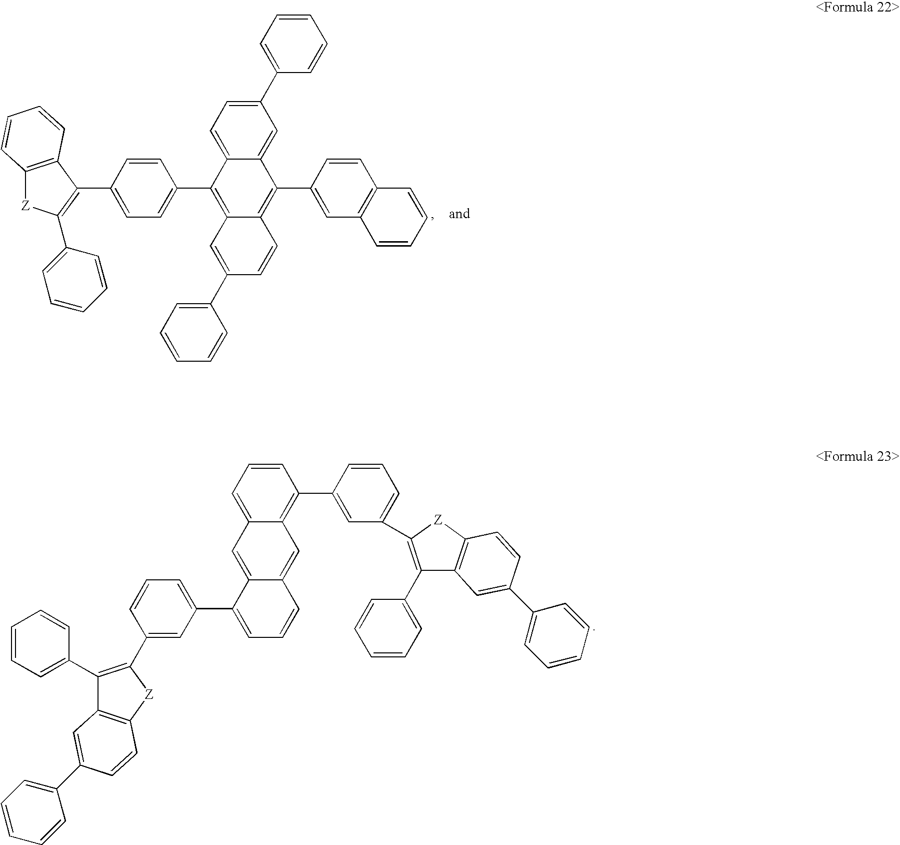

The compound represented by Formula 1 according to the embodiment of the present invention can be synthesized by using a known synthesis method, and the synthetic pathway of the compound is shown in detail in Reaction Schemes of Synthesis Example 1 which will be described later.

An organic light emitting device according to an embodiment of the present invention includes a first electrode; a second electrode; and at least one organic layer interposed between the first electrode and the second electrode, wherein the organic layer includes the compound represented by Formula 1 according to the embodiment of the present invention. Specifically, in the organic light emitting device according to the embodiment of the present invention, the compound represented by Formula 1 may be any one of the compounds represented by Formulae 3 through 23.

The organic layer may be formed by wet spinning or thermal transferring, such as spin coating, inkjet printing, or spray printing. However, the organic layer can also be formed by other methods.

The compound represented by Formula 1 may efficiently function as a hole injection layer forming material, a hole transport layer forming material, an electron blocking layer forming material, a hole blocking layer forming material, an electron transport layer forming material, an electron injection layer forming material, or a host or dopant material of an emission layer. According to an embodiment of the present invention, the emission layer may further include, in addition to the compound according to the present invention, other light emitting materials.

The organic light emitting device according to the present invention may have various structures. The organic light emitting device according to an embodiment of the present invention may further include, between the first electrode and the second electrode, at least one layer selected from the group consisting of a hole injection layer, a hole transport layer, a hole blocking layer, an electron blocking layer, an electron transport layer and an electron injection layer.

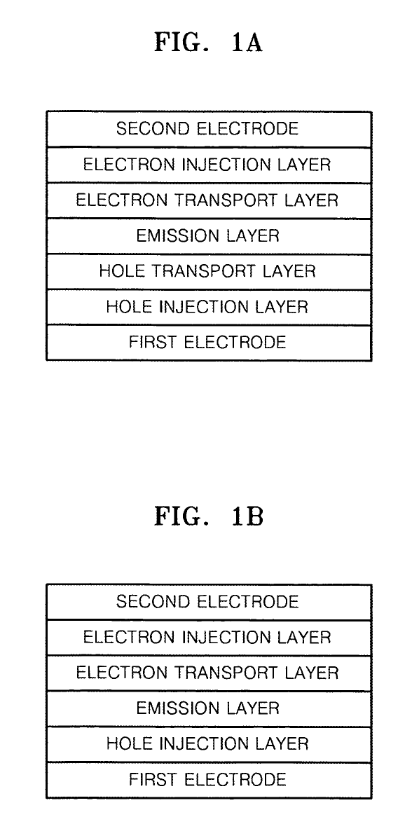

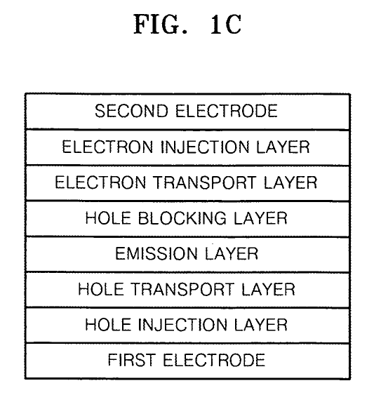

FIGS. 1A through 1C illustrate schematic stacked structures of organic light emitting devices according to embodiments of the present invention. The organic light emitting device illustrated in FIG. 1A has a structure of first electrode/hole injection layer/hole transport layer/emission layer/electron transport layer/electron injection layer/second electrode. The organic light emitting device illustrated in FIG. 1B has a structure of first electrode/hole injection layer/emission layer/electron transport layer/electron injection layer/second electrode. The organic light emitting device illustrated in FIG. 1C has a structure of first electrode/hole injection layer/hole transport layer/emission layer/hole blocking layer/electron transport layer/electron injection layer/second electrode. In these structures, at least one layer selected from the group consisting of the electron injection layer, the electron transport layer, the hole blocking layer, the emission layer, the hole injection layer and the hole transport layer may include the compound of Formula 1.

The emission layer of an organic light emitting device according to an embodiment of the present invention may further include a red, green, blue, or white phosphorescent or fluorescent dopant. Among those dopants, the red, green, blue, or white fluorescent dopant may be an organometallic compound including at least one element selected from the group consisting of Ir, Pt, Os, Ti, Zr, Hf, Eu, Tb and Tm.

Since the compound of Formula 1 has excellent color purity, color stability, and excellent thermal stability, the compound can also be used as a display material for mobile phones, MP3 players, PDAs, digital cameras, car appliances, or TVs. In addition, the compound according to the present invention can be used as an organic conductive material, or an electron material of a solar cell.

An exemplary method of manufacturing an organic light emitting device according to the present invention will now be described in detail with reference to the organic light emitting device of FIG. 1C.

First, a first electrode forming material having a high work function is applied to a substrate by deposition or sputtering, thereby forming a first electrode. The first electrode may function as an anode. The substrate may be any substrate that is used in conventional organic light emitting devices. For example, the substrate may be a glass substrate, or a transparent plastic substrate having high mechanical strength, high thermal stability, high transparency, a planar surface, convenience for handling, and excellent waterproof properties. The first electrode forming material may be a transparent and highly conductive material, such as indium tin oxide (ITO), indium zinc oxide (IZO), tin oxide (SnO₂), or zinc oxide (ZnO).

Then, a hole injection layer (HIL) can be formed on the first electrode by using a vacuum deposition method, a spin coating method, a casting method, or a Langmuir Blodgett (LB) method.

When the HIL is formed using a vacuum deposition method, the deposition conditions may differ according to the type of a HIL forming material and the structure and thermal properties of the desired HIL. For example, the deposition temperature may be in a range of 100 to 500° C., the degree of vacuum may be in a range of 10⁻⁸ to 10⁻³ torr, the deposition rate may be in a range of 0.01 to 100 Å/sec, and the thickness of the HIL may be in a range of 10 Å to 5 μm.

When the HIL is formed using a spin coating method, the coating conditions may differ according to the type of a HIL forming material and the structure and thermal properties of the desired HIL. For example, the coating rate may be in a range of about 2000 rpm to 5000 rpm, and a heat treatment temperature at which a solvent used in the coating process is removed may be in a range of about 80° C. to 200° C.

The HIL forming material may be the compound represented by Formula 1, 2a, or 2b. In addition, the HIL forming material may be any known hole injecting material, such as: a copper phthalocyanine compound disclosed in U.S. Pat. No. 4,356,429 which is incorporated herein by reference; a star-burst type amine derivative, such as TCTA, m-MTDATA, or m-MTDAPB, disclosed in Advanced Material, 6, p. 677 (1994) which is incorporated herein by reference; or a soluble and conductive polymer, such as polyaniline/dodecylbenzenesulfonic acid (Pani/DBSA), poly(3,4-ethylenedioxythiophene)/poly(4-styrenesulfonate (PEDOT/PSS)), polyaniline/camphor sulfonicacid (Pani/CSA), or polyaniline/poly(4-styrenesulfonate) (PANI/PSS).

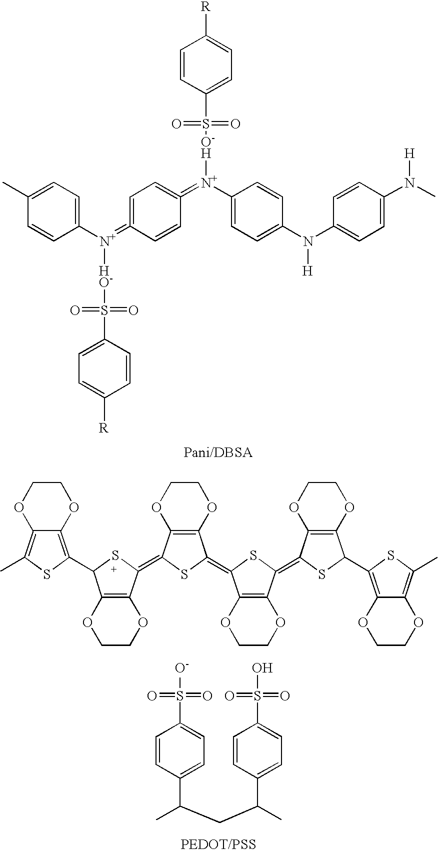

The thickness of the HIL may be in a range of about 100 Å to 10,000 Å, specifically 100 Å to 1000 Å. If the thickness of the HIL is less than 100 Å, hole injecting properties may be degraded. On the other hand, if the thickness of the HIL is greater than 10,000 Å, the driving voltage of the organic light emitting device may be increased.

Then, a hole transport layer (HTL) can be formed on the HIL by using a vacuum deposition method, a spin coating method, a casting method, or an LB method. When the HTL is formed by using a vacuum deposition method or a spin coating method, the deposition and coating conditions may differ according to the type of a HTL forming material, and can be very similar to the deposition and coating conditions used to form the HIL.

The HTL forming material may be the compound represented by Formula 1. The HTL forming material may also be any known hole transporting material. In this regard, the HTL forming material may be a carbazole derivative, such as N-phenylcarbazole or polyvinylcarbazole; or a conventional amine derivative having an aromatic condensation ring, such as N,N′-bis(3-methylphenyl)-N,N′-diphenyl-[1,1-biphenyl]-4,4′-diamine (TPD), or N,N′-di(naphtalene-1-yl)-N,N′-diphenyl benzidine (α-NPD).

The thickness of the HTL may be in a range of about 50 Å to 1000 Å, specifically 100 Å to 600 Å. If the thickness of the HTL is less than 50 Å, hole transporting properties may be degraded. On the other hand, if the thickness of the HTL is greater than 1000 Å, the driving voltage of the organic light emitting device may be increased.

Then, an emission layer (EML) may be formed on the HTL by using a vacuum deposition method, a spin coating method, a casting method, or an LB method. When the EML is formed by using a vacuum deposition method or a spin coating method, the deposition and coating conditions may differ according to the type of an EML forming material, and can be very similar to the deposition and coating conditions used to form the HIL.

The EML forming material may be the compound represented by Formula 1 according to the present invention. According to an embodiment of the present invention, the EML forming material can include the compound represented by Formula 1 and an organic semiconducting material. Examples of the organic semiconducting material include pentacene, polythiophene, and tetrathiafulvalene.

According to another embodiment of the present invention, the compound represented by Formula 1 may be used together with a host. The host may be, for example, Alq₃, 4,4′-N,N′-dicarbazole-biphenyl (CBP), or poly(n-vinylcarbazole) (PVK).

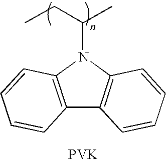

According to an embodiment of the present invention, the EML may include, in addition to the compound represented by Formula 1 any known dopant, such as a fluorescent or phosphorescent dopant. Examples of the fluorescent dopant include IDE102 and IDE105 (manufactured by Idemitsu Kosan Co., Ltd.) and C545T (manufactured by Hayashibara Co., Ltd.). The phosphorescent dopant can be categorized into a red phosphorescent dopant, a green phosphorescent dopant, and a blue phosphorescent dopant. The red phosphorescent dopant may be PtOEP, or RD 61 (manufactured by UDC Co.). The green phosphorescent dopant may be Ir(PPy)3 where PPy denotes 2-phenylpyridine. The blue phosphorescent dopant may be F2Irpic.

The doping concentration may be not limited and may be in a range of 0.01-15 parts by weight based on 100 parts by weight of the host.

The thickness of the EML may be in a range of about 100 Å to 1000 Å, specifically 200 Å to 600 Å, but is not limited thereto. If the thickness of the EML is less than 100 Å, emitting properties of the EML may be degraded. On the other hand, if the thickness of the EML is greater than 1000 Å, the driving voltage of the organic light emitting device may be increased.

However, when the EML is formed using a phosphorescent dopant, triplet excitons or holes can diffuse toward an electron transport layer. The diffusion of triplet excitons or holes can be prevented by forming a hole blocking layer (HBL) on the EML using a vacuum deposition method, a spin coating method, a casting method, or a LB method. When the HBL is formed by using a vacuum deposition method or a spin coating method, the deposition and coating conditions may differ according to the type of the HBL forming material, and can be very similar to the deposition and coating conditions used to form the HIL. The HBL forming material may be, for example, an oxadiazole derivative, a triazole derivative, a phenanthroline derivative, a hole blocking material disclosed in JP 11-329734(A1) which is incorporated herein by reference, or bathocuproine (BCP).

The thickness of the HBL may be in a range of about 50 Å to 1000 Å, specifically 100 Å to 300 Å. If the thickness of the HBL is less than 50 Å, hole blocking properties may be degraded. On the other hand, if the thickness of the HBL is greater than 1000 Å, the driving voltage of the organic light emitting device may be increased.

Then, an electron transport layer (ETL) can be formed on the HBL by using a vacuum deposition method, a spin coating method, or a casting method. When the ETL is formed by using a vacuum deposition method or a spin coating method, the deposition and coating conditions may differ according to the type of the ETL forming material and can be very similar to the deposition and coating conditions used to form the HIL. The ETL forming material may be any known material that stably transports electrons injected from an electron injection electrode, that is, a cathode. For example, the ETL forming material may be a quinoline derivative, such as tris(8-quinolinorate)aluminum (Alq₃), 3-phenyl-4-(1′-naphthyl)-5-phenyl-1,2,4-triazole (TAZ) or aluminum(III)bis(2-methyl-8-quinolinato)4-phenylphenolate (Balq).

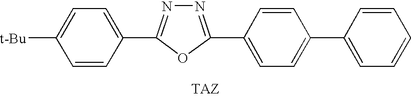

The thickness of the ETL may be in a range of about 100 Å to 1000 Å, specifically 200 Å to 500 Å. If the thickness of the ETL is less than 100 Å, electron transporting properties may be degraded. On the other hand, if the thickness of the ETL is greater than 1000 Å, the driving voltage of the organic light emitting device may be increased.

In addition, an electron injection layer (EIL) forming material that allows electrons to be easily injected from the cathode can be deposited on the ETL to form an EIL, and is not limited.

The EIL forming material may be any known EIL forming material, such as LiF, NaCl, CsF, Li₂O, or BaO. The deposition conditions of the ETL may differ according to the type of the EIL forming material and can be very similar to the deposition and coating conditions used to form the HIL.

The thickness of the EIL may be in a range of about 1 Å to 100 Å, specifically 5 Å to 50 Å. If the thickness of the EIL is less than 1 Å, electron injection properties may be degraded. On the other hand, if the thickness of the EIL is greater than 100 Å, the driving voltage of the organic light emitting device may be increased.

Finally, a second electrode can be formed on the EIL by using a vacuum deposition method or a sputtering method. The second electrode may act as a cathode. A second electrode forming material may be a metal, an alloy, an electroconductive compound, or a mixture thereof, each having a low work function. For example, the second electrode forming metal may be Li, Mg, Al, Al—Li, Ca, Mg—In, or Mg—Ag. If the organic light emitting device is a top-emission type light emitting device, the second electrode can be a transmissive cathode formed of ITO or IZO.

An organic light emitting device according to an embodiment of the present invention can also include, in addition to the structure of first electrode/HIL/HTL/EML/HBL/ETL/EIL/second electrode illustrated in FIG. 1C, other structures. When required, the layers described above may be selectively not used.

The present invention will be described in further detail with reference to the following examples. These examples are for illustrative purposes only and are not intended to limit the scope of the present invention.

### SYNTHESIS EXAMPLE 1

A compound represented by Formula 3 was synthesized according to the pathway of Reaction Scheme 1.

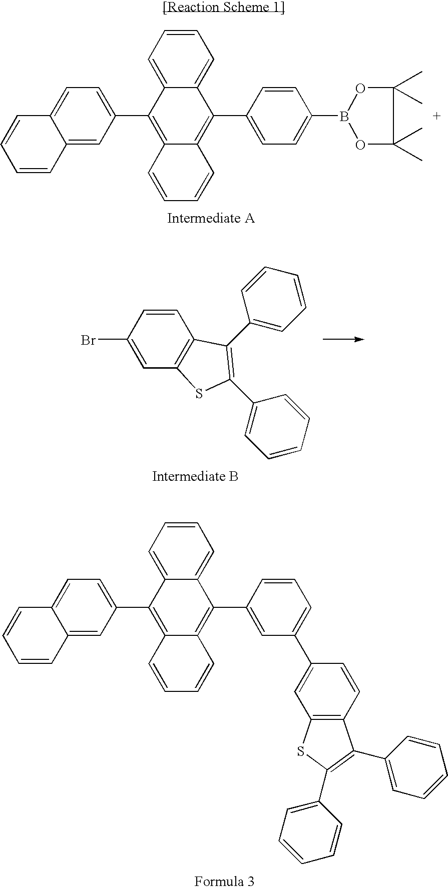

3 g (8.21 mmol) Intermediate B was dissolved in 70 ml of THF. Separately, 4.99 g (9.85 mmol) of Intermediate A, 470 mg (0.4 mmol) of tetrakis(triphenylphoshpine)paladium (0), and 0.82 mmol of K₂CO₃ were dissolved in 30 ml of toluene and 8.4 ml of water and then added to the obtained Intermediate B solution. The resultant solution was stirred for 24 hours at a reflux temperature, cooled to room temperature, and then 100 ml of diethylether was added thereto. Then, the resultant solution was washed twice with 100 ml of water and an organic layer was collected and dried over anhydrous magnesium sulfate. Then, the solvent used was evaporated to obtain a pre-product. The pre-product was refined by silicagel column chromatography and the refined product was re-crystallized to obtain 400 mg (Yield: 63%) of Compound 3 that is a compound represented by Formula 3.

1H-NMR (CDCl₃, 300 MHz, ppm): 7.98-7.08 (m, 32H)

Glass transition temperature (Tg), a melting point (Tm), thermal degradation temperature (Td) and highest occupied molecular orbit (HOMO) of Compound 3 were measured. The results are shown in Table 1.

TABLE 1

| | Tg (° C.) | Tm (° C.) | Td (° C.) | HOMO |
|---|---|---|---|---|
| Compound 3 | 150 | 335 | 415 | 5.82 |

Evaluation 1: Emission Properties

The photoluminescence (PL) of Compound 3 in a film state and a solution state was measured to evaluate emission properties of Compound 3.

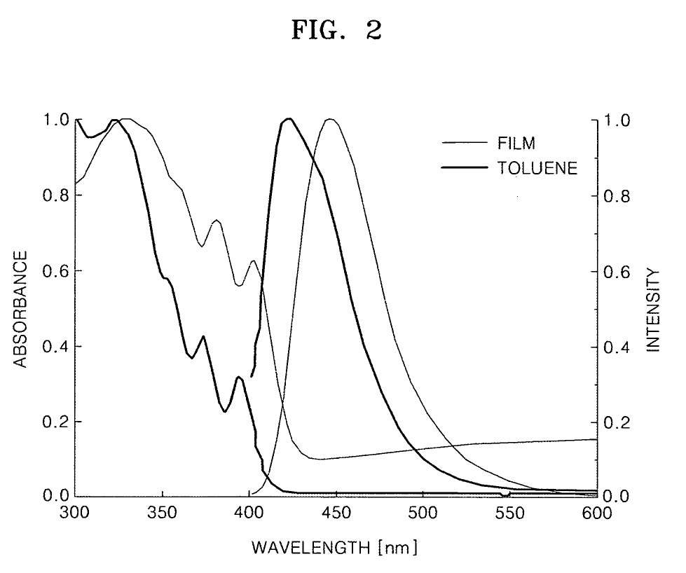

To evaluate optical properties of Compound 3 in a solution state, Compound 3 was diluted with toluene such that the concentration of the resultant solution was 10 mM, and then a PL spectrum of the obtained solution was measured with an ISC PC1 spectrofluorometer equipped with a Xenon lamp. This experiment was repeatly performed using Compound 3. The results are shown in FIG. 2.

To evaluate optical properties of Compound 3 in a film state, a quartz substrate was washed with acetone and pure water. Then, Compound 3 was spin-coated on the substrate, and then heat-treated at 110° C. for 30 minutes to form a film having a thickness of 1,000 Å. The PL spectrum of the obtained film was measured. This experiment was repeatly performed using the compound represented by Formula 3.

FIG. 2 illustrates an absorption spectrum of Compound 3 synthesized according to Synthesis Example 1, showing solution properties and film processibility.

Therefore, it can be seen that the compound according to the present invention has appropriate emission properties sufficient for use in an organic light emitting device.

### Example 1

An organic light emitting device having the following structure was manufactured using Compound 3 as a host and DPAVBi as a dopant: ITO(1000 Å)/(M-TDATA)(35 nm)/α-NPD(30 nm)/(95 wt. % of Compound 3 /5 wt. % of DPAVBi)(35 nm)/ALq₃(18 nm)/LiF(0.7 nm)/Al(150 nm).

A 15 Ω/cm² (1000 Å) ITO glass substrate was cut to a size of 50 mm×50 mm×0.7 mm and then was sonificated in aceton isopropyl alcohol and pure water each for 15 minutes and washed with UV and ozone for 30 minutes, thereby forming an anode. M-TDATA was vacuum deposited on the anode to a thickness of 35 nm and α-NPD was vacuum deposited thereon to a thickness of 30 nm to form a hole injection layer (HIL). Then, Compound 3 and DPAVBi were vacuum deposited on the HIL in a ratio of 95 wt. % to 5 wt. % to form an EML having a thickness of 35 nm. Then, ALq₃ was vacuum deposited on the EML to form an ETL having a thickness of 18 nm. Then, LiF was vacuum deposited on the ETL to form an EIL having a thickness of 0.7 nm and Al was vacuum deposited thereon to form a cathode having a thickness of 150 nm, thereby completing the manufacture of an organic light emitting device having the structure illustrated in FIG. 1B. Properties of the organic light emitting device are shown in Table 2 below.

TABLE 2

| | Driving | Current | | |
|---|---|---|---|---|
| | voltage | efficiency | Current | CIE color |
| | (V) | lm/W | efficiency | coordinate |
| | (Op.V @ | @ 1000 nit | (cd/A) | (@ |
| Compound | 1000 nit) | (max. @V) | (max. @V) | 1000 nit) |
| 3 | 3.6/6.6 | 3.03 | 6.55 | (0.14, 0.16) |
| | | (4.36 @4.4 V) | (6.55 @6.4 V) | |

FIG. 2 illustrates UV and PL spectra of Compound 3 showing properties of Compound 3 in a solution state and in a film state.

Accordingly, it can be seen that an organic light emitting device including an EML manufactured using the compound according to the present invention has excellent EL emission properties suitable for phosphoresent and fluorescent material.

A compound represented by Formula 1 according to the present invention can be used to form a thermally stable organic layer, and thus, an organic light emitting device including the thermally stable organic layer has low driving voltage, high brightness, high efficiency, and high color purity.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A compound represented by Formula 1:
X—Ar₁—Ar₂—Y (1)
where Ar₁ is a substituted or unsubstituted C₅-C₅₀ arylene group, a substituted or unsubstituted C₅-C₃₀ heteroarylene group, a substituted or unsubstituted C₅-C₅₀ alkenylene group, or a combination thereof connected via a single bond;
Ar₂ is a substituted or unsubstituted C₅-C₁₅ arylene group, a substituted or unsubstituted C₅-C₁₅ heteroarylene group, a substituted or unsubstituted C₅-c₁₅ alkenylene group, a substituted or unsubstituted C₅-c₁₅ cycloalkylene group, or a combination thereof by a single bond;
X is each independently hydrogen, halogen, a cyano group, a hydroxyl group, a substituted or unsubstituted C₁-C₂₀ alkyl group, a substituted or unsubstituted C₃-C₂₀ cycloalkyl group, a substituted or unsubstituted C₅-C₂₀ heterocycloalkyl group, a substituted or unsubstituted C₁-C₂₀ alkoxy group, a substituted or unsubstituted C₆-C₃₀ aryl group, a substituted or unsubstituted C₆-C₃₀ arylalkyl group, a substituted or unsubstituted C₂-C₃₀ heteroaryl group, a substituted or unsubstituted C₃-C₃₀ aryl amino group, a substituted or unsubstituted C₃-C₃₀ arylsilane group, a group represented by Formula 2a or a group represented by Formula 2b; and
Y is a group represented by Formula 2a or Formula 2b: 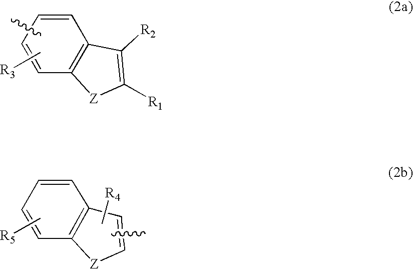
where Z is S, O, N, Se, or SO₂;
R₁ through R₅ are each independently hydrogen, halogen, a cyano group, a hydroxyl group, a substituted or unsubstituted C₁-C₂₀ alkyl group, a substituted or unsubstituted C₃-C₂₀ cycloalkyl group, a substituted or unsubstituted C₅-C₂₀ heterocycloalkyl group, a substituted or unsubstituted C₁-C₂₀ alkoxy group, a substituted or unsubstituted C₆-C₃₀ aryl group, a substituted or unsubstituted C₆-C₃₀ arylalkyl group, a substituted or unsubstituted C₂-C₃₀ heteroaryl group, a substituted or unsubstituted C₃-C₃₀ aryl amino group, or a substituted or unsubstituted C₃-C₃₀ arylsilane group; and [symbol] denotes a linking group.

2. The compound of claim 1, wherein Ar₁ is represented by ₒne selected from the groups consisting of the following Formulae: 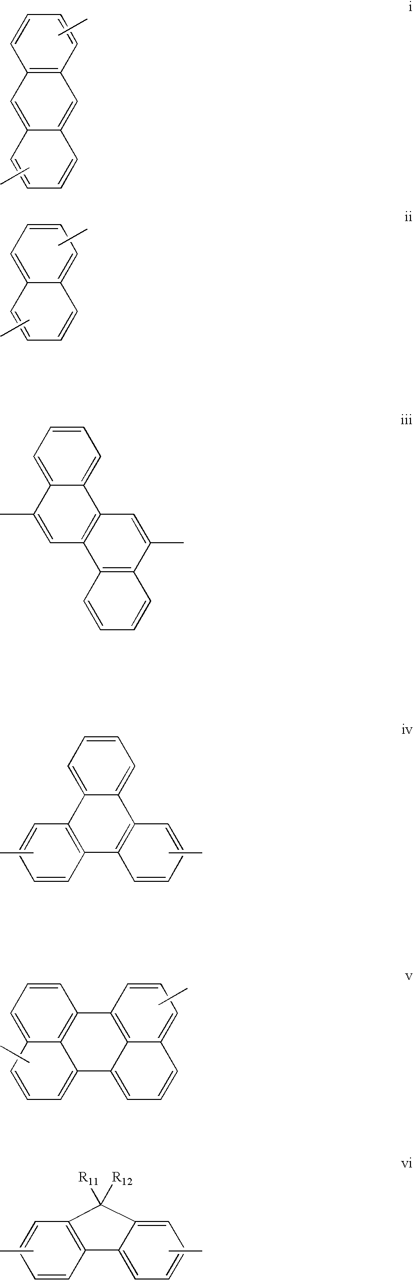 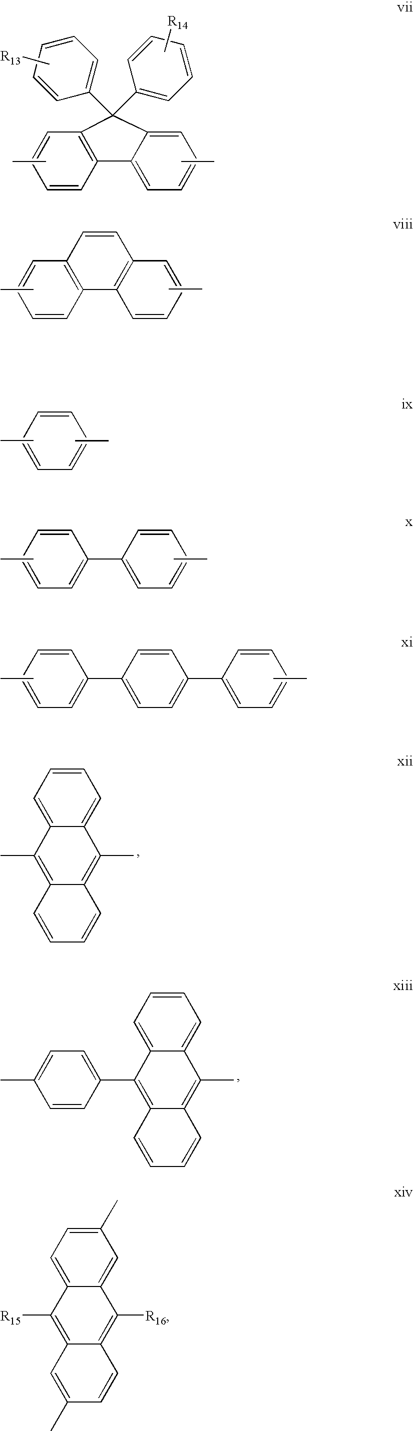 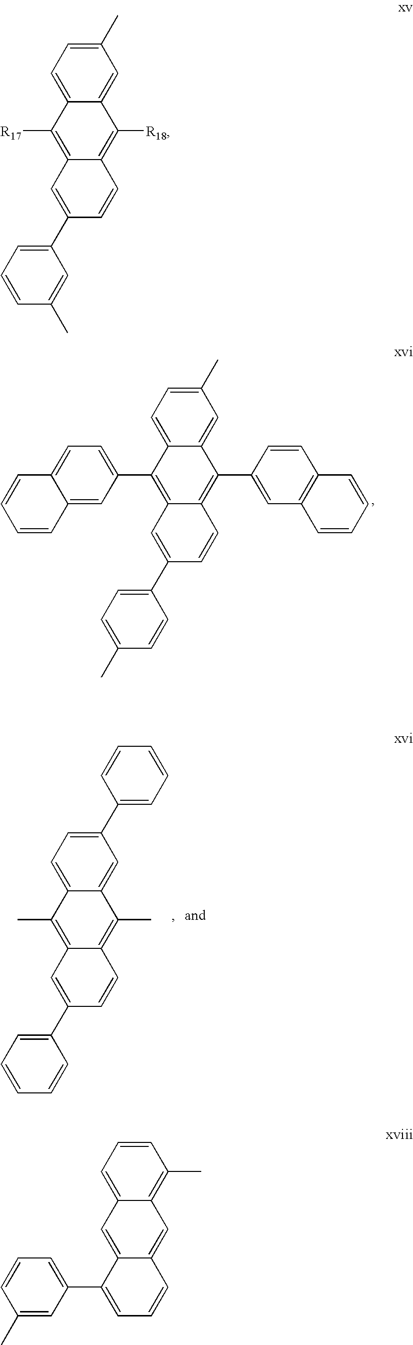
where R₁₁, R₁₂, R₁₃, R₁₄, R₁₅, R₁₆, R₁₇ and R₁₈ are each independently hydrogen, a substituted or unsubstituted C₁-C₂₀ alkyl group, a substituted or unsubstituted C₆-C₃₀ aryl group, a substituted or unsubstituted C₂-C₃₀ heteroaryl group, a substituted or unsubstituted C₅-C₂₀ cycloalkyl group or a substituted or unsubstituted C₅-C₃₀ heterocycloalkyl group.

3. The compound of claim 1, wherein Ar₂ has one of the following
structures:

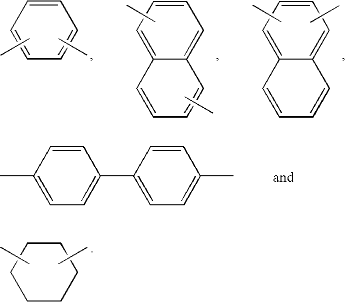

4. The compound of claim 1, wherein, R₁ through R₅, and X are each independently hydrogen, a C₁-C₂₀ alkyl group, a C₁-C₂₀ alkoxy group, a phenyl group, a biphenyl group, a pentalenyl group, an indenyl group, a naphthyl group, a biphenylenyl group, an anthracenyl group, an azurenyl group, a heptalenyl group, an acenaphthylenyl group, a phenalenyl group, a fluorenyl group, a methylanthryl group, a phenanthrenyl group, a triphenylenyl group, a pyrenyl group, a chrysenyl group, an ethyl-chrysenyl group, a picenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a fluorenyl group, a pyranthrenyl group, an ovalenyl group, a carbazolyl group, a thiophenyl group, an indolyl group, a purinyl group, a benzimidazolyl group, a quinolinyl group, a benzothiophenyl group, a parathiazinyl group, a pyrrolyl group, a pyrazolyl group, an imidazolyl group, an imidazolinyl group, an oxazolyl group, a thiazolyl group, a triazolyl group, a tetrazolyl group, an oxadiazolyl group, a pyridinyl group, a pyridazinyl group, a pyrimidinyl group, a pyrazinyl group, a thianthrenyl group, a cyclopentyl group, a cyclohexyl group, an oxiranyl group, a pyrrolidinyl group, a pyrazolidinyl group, an imidazolyl group, a piperidinyl group, a piperazinyl group, a morpholinyl group, a di(C₆-C₅₀ aryl)amino group, a tri(C₆-C₅₀aryl)silyl group, or derivatives thereof.

5. The compound of claim 1, wherein the compound represented by Formula 1 is any one compound selected from the compounds represented by Formulae 3 to 7:

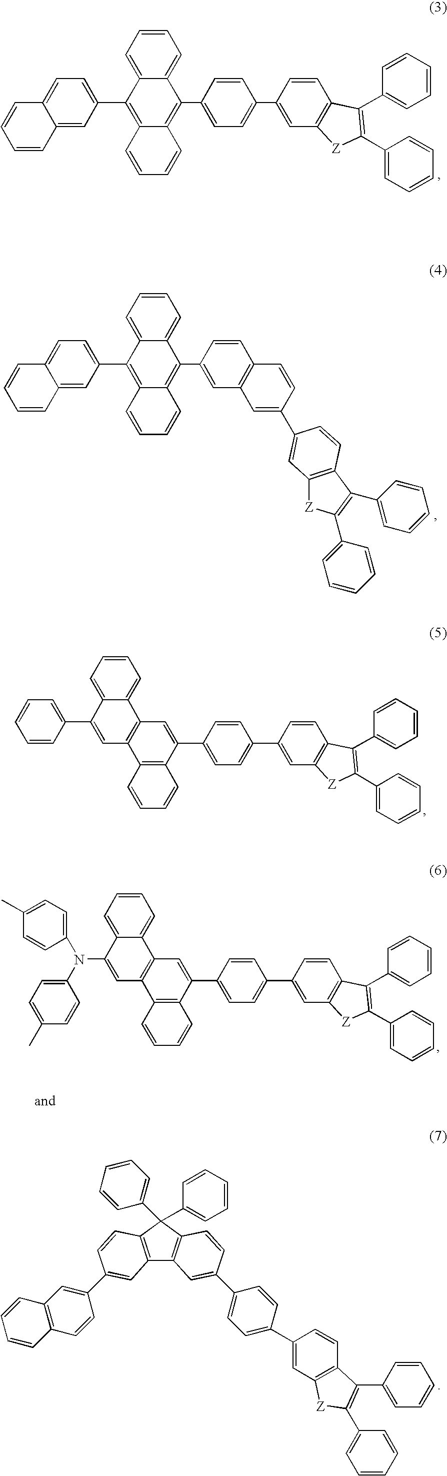

6. The compound of claim 1, wherein the compound represented by Formula 1 is any one compound selected from the compounds represented by Formulae 8 to 12:

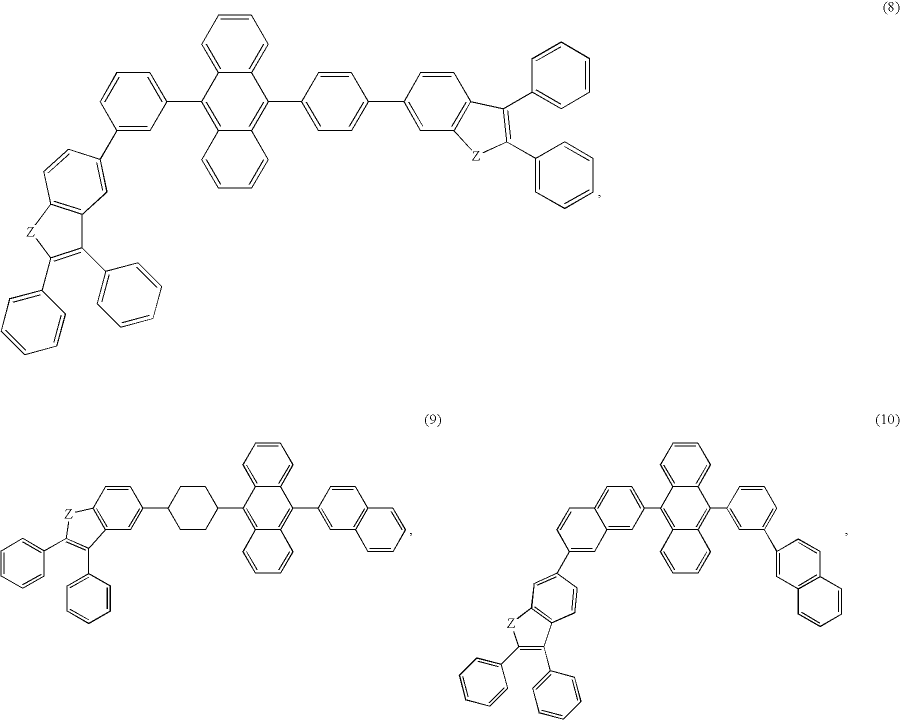

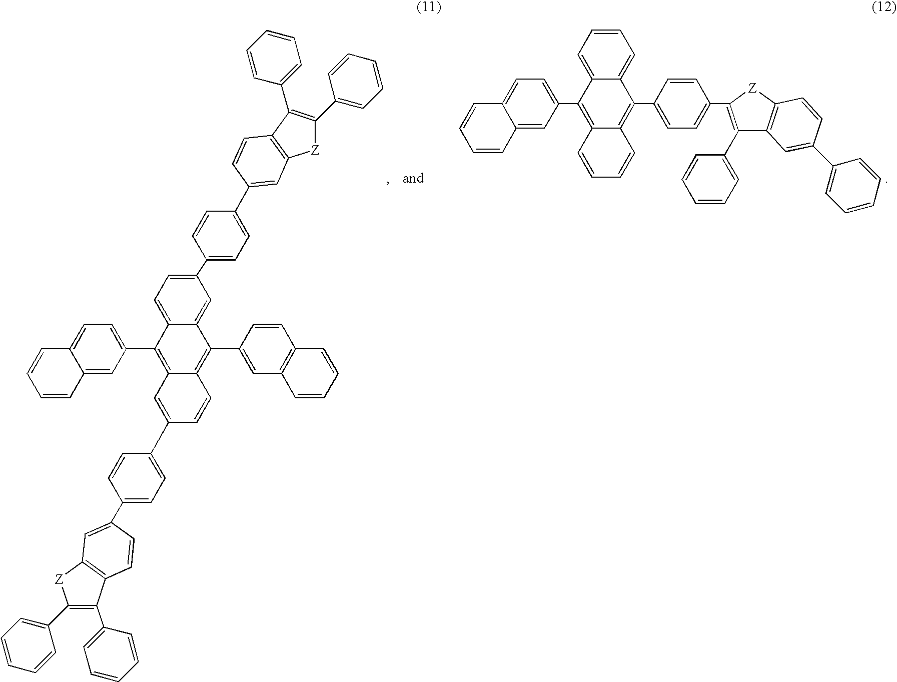

7. The compound of claim 1, wherein the compound represented by Formula 1 is any one compound selected from the compounds represented by Formulae 13 to 17:

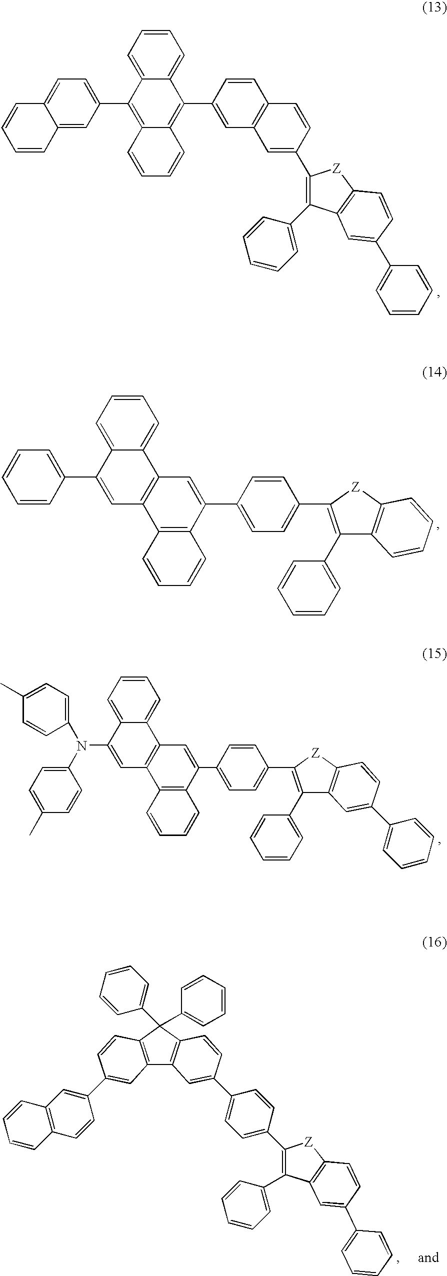

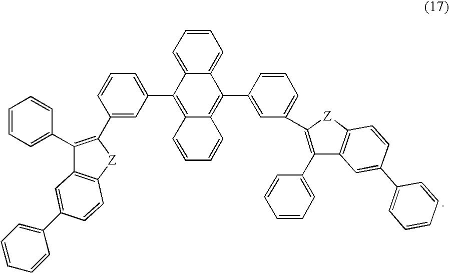

8. The compound of claim 1, wherein the compound represented by Formula 1 is any one compound selected from the compounds represented by Formulae 18 to 23:

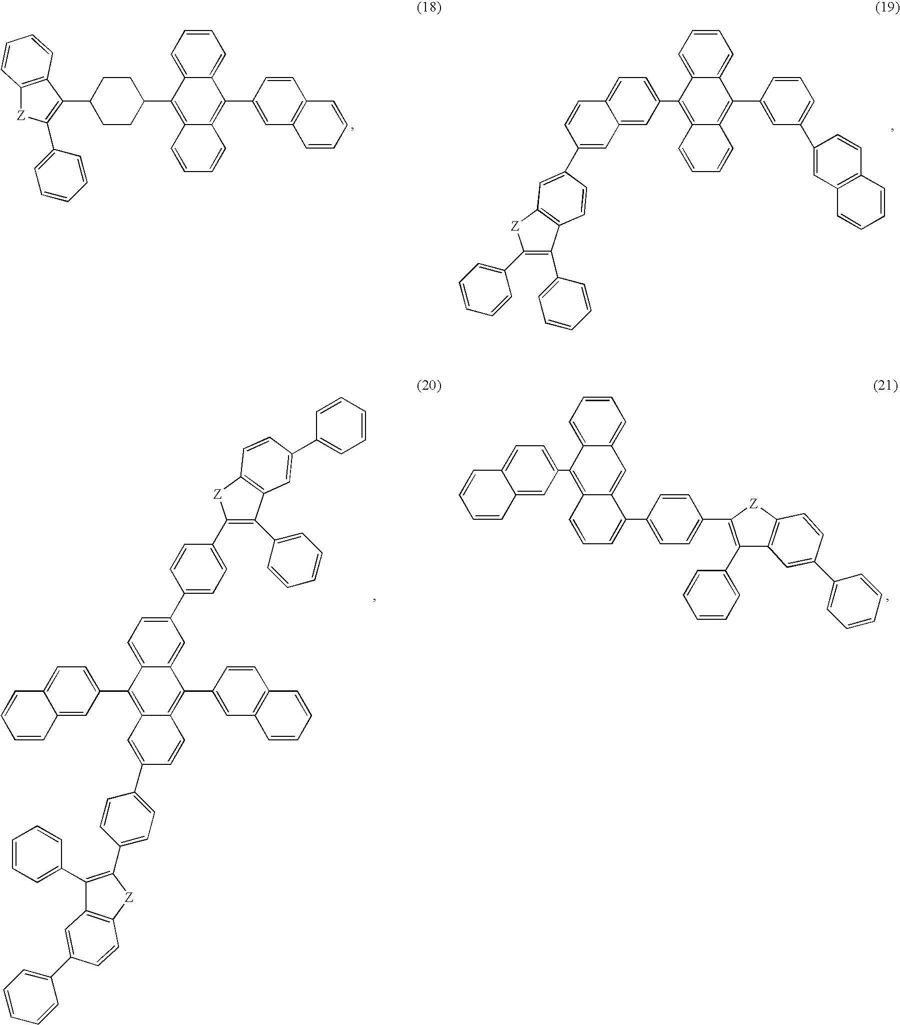

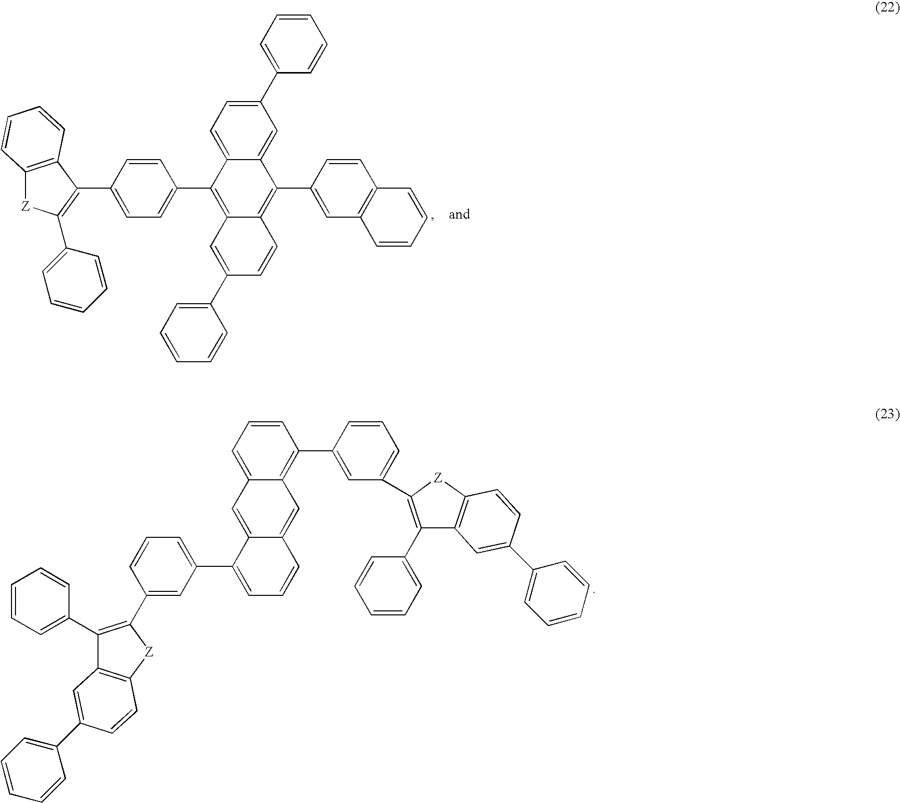

9. An organic light emitting device having an organic layer comprising the compound of claim 1.

10. An organic light emitting device comprising:
a first electrode;
a second electrode; and
at least one organic layer interposed between the first electrode and the second electrode, said at least one organic layer comprising an organic layer comprised of a compound represented by Formula 1:
X—Ar₁—Ar₂—Y (1)
where Ar₁ is a substituted or unsubstituted C₅-C₅₀ arylene group, a substituted or unsubstituted C₅-C₃₀ heteroarylene group, a substituted or unsubstituted C₅-C₅₀ alkenylene group, or a combination thereof connected via a single bond;
Ar₂ is a substituted or unsubstituted C₅-C₁₅ arylene group, a substituted or unsubstituted C₅-C₁₅ heteroarylene group, a substituted or unsubstituted C₅-C₁₅ alkenylene group, a substituted or unsubstituted C₅-C₁₅ cycloalkylene group, or a combination thereof by a single bond;
X is each independently hydrogen, halogen, a cyano group, a hydroxyl group, a substituted or unsubstituted C₁-C₂₀ alkyl group, a substituted or unsubstituted C₃-C₂₀ cycloalkyl group, a substituted or unsubstituted C₅-C₂₀ heterocycloalkyl group, a substituted or unsubstituted C₁-C₂₀ alkoxy group, a substituted or unsubstituted C₆-C₃₀ aryl group, a substituted or unsubstituted C₆-C₃₀ arylalkyl group, a substituted or unsubstituted C₂-C₃₀ heteroaryl group, a substituted or unsubstituted C₃-C₃₀ aryl amino group, a substituted or unsubstituted C₃-C₃₀ arylsilane group, a group represented by Formula 2a or a group represented by Formula 2b; and
Y is a group represented by Formula 2a or Formula 2b: 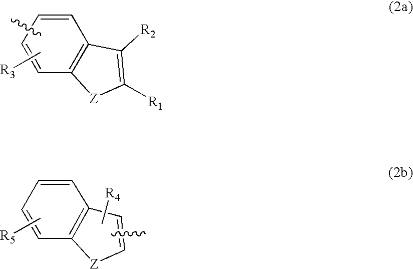
where Z is S, O, N, Se, or SO₂;
R₁ through R₅ are each independently hydrogen, halogen, a cyano group, a hydroxyl group, a substituted or unsubstituted C₁-C₂₀ alkyl group, a substituted or unsubstituted C₃-C₂₀ cycloalkyl group, a substituted or unsubstituted C₅-C₂₀ heterocycloalkyl group, a substituted or unsubstituted C₁-C₂₀ alkoxy group, a substituted or unsubstituted C₆-C₃₀ aryl group, a substituted or unsubstituted C₆-C₃₀ arylalkyl group, a substituted or unsubstituted C₂-C₃₀ heteroaryl group, a substituted or unsubstituted C₃-C₃₀ aryl amino group, or a substituted or unsubstituted C₃-C₃₀ arylsilane group; and 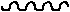
denotes a linking group.

11. The organic light emitting device of claim 10, wherein the organic layer comprised of the compound represented by Formula 1 is selected from the group consisting of a hole injection layer, a hole transport layer, an electron blocking layer, an emission layer, a hole blocking layer, an electron transport layer and an electron injection layer.

12. The organic light emitting device of claim 10, further comprising, between the first electrode and the second electrode, at least one layer selected from the group consisting of a hole injection layer, a hole transport layer, an electron blocking layer, a hole blocking layer, an electron transport layer and an electron injection layer.

13. The organic light emitting device of claim 11, wherein, when the organic layer comprised of the compound represented by Formula 1 is the emission layer, and the emission layer further comprises a red, green, or white phosphorescent or fluorescent dopant.

14. The organic light emitting device of claim 13, wherein the phosphorescent dopant comprises an organometallic compound comprising at least one element selected from the group consisting of Ir, Pt, Os, Ti, Zr, Hf, Eu, Tb and Tm.

15. The organic light emitting device of claim 10, wherein Ar₁ is represented by one selected from the groups consisting of the following Formulae: 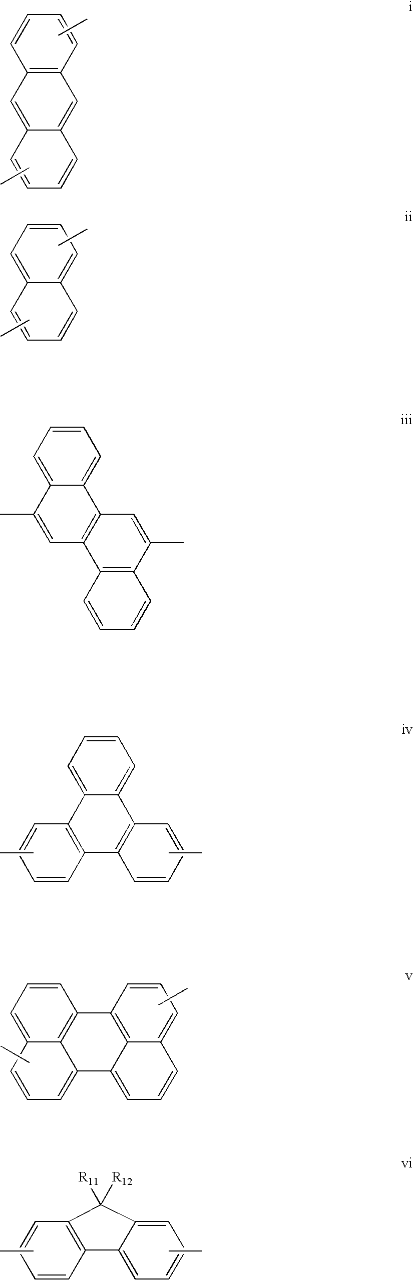 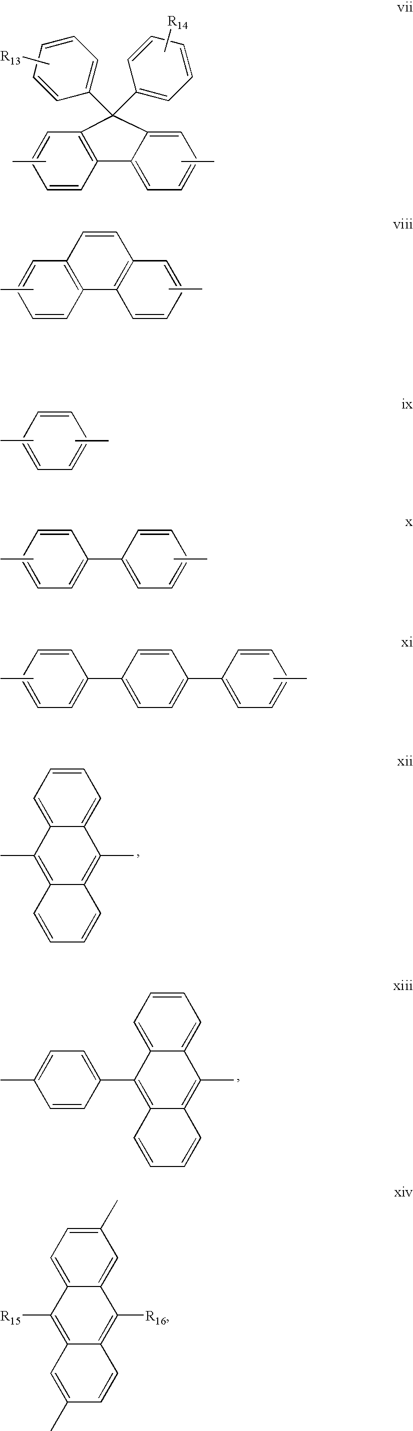 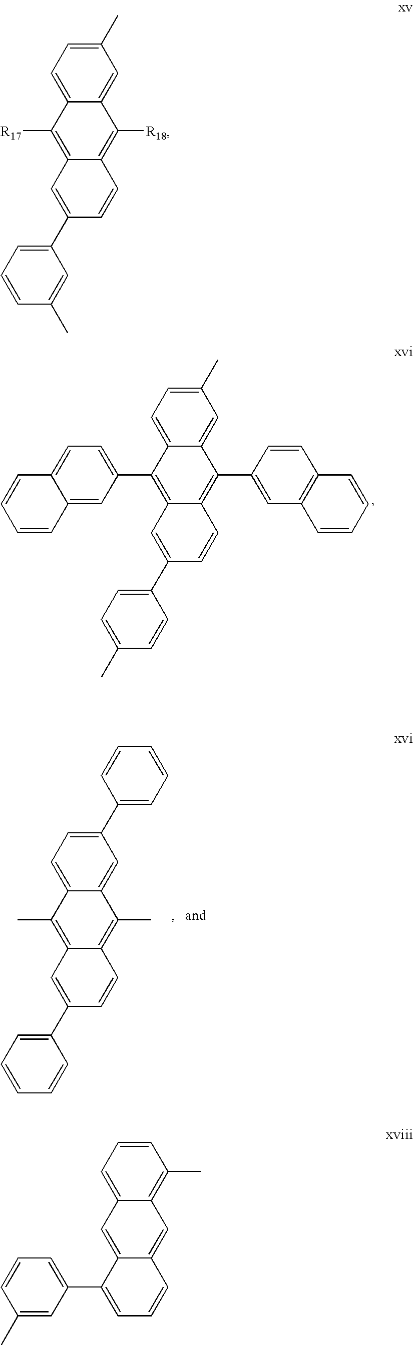
where R₁₁, R₁₂, R₁₃, R₁₄, R₁₅, R₁₆, R₁₇ and R₁₈ are each independently hydrogen, a substituted or unsubstituted C₁-C₂₀ alkyl group, a substituted or unsubstituted C₆-C₃₀ aryl group, a substituted or unsubstituted C₂-C₃₀ heteroaryl group, a substituted or unsubstituted C₅-C₂₀ cycloalkyl group or a substituted or unsubstituted C₅-C₃₀ heterocycloalkyl group.

16. The organic light emitting device of claim 10, wherein Ar₂ has one of the 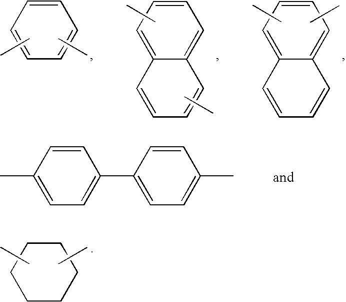
following structures:

17. The organic light emitting device of claim 10, wherein, R₁ through R₅, and X are each independently hydrogen, a C₁-C₂₀ alkyl group, a C₁-C₂₀ alkoxy group, a phenyl group, a biphenyl group, a pentalenyl group, an indenyl group, a naphthyl group, a biphenylenyl group, an anthracenyl group, an azurenyl group, a heptalenyl group, an acenaphthylenyl group, a phenalenyl group, a fluorenyl group, a methylanthryl group, a phenanthrenyl group, a triphenylenyl group, a pyrenyl group, a chrysenyl group, an ethyl-chrysenyl group, a picenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a fluorenyl group, a pyranthrenyl group, an ovalenyl group, a carbazolyl group, a thiophenyl group, an indolyl group, a purinyl group, a benzimidazolyl group, a quinolinyl group, a benzothiophenyl group, a parathiazinyl group, a pyrrolyl group, a pyrazolyl group, an imidazolyl group, an imidazolinyl group, an oxazolyl group, a thiazolyl group, a triazolyl group, a tetrazolyl group, an oxadiazolyl group, a pyridinyl group, a pyridazinyl group, a pyrimidinyl group, a pyrazinyl group, a thianthrenyl group, a cyclopentyl group, a cyclohexyl group, an oxiranyl group, a pyrrolidinyl group, a pyrazolidinyl group, an imidazolyl group, a piperidinyl group, a piperazinyl group, a morpholinyl group, a di(C₆-C₅₀ aryl)amino group, a tri(C₆-C₅₀aryl)silyl group, or derivatives thereof.

18. The organic light emitting device of claim 10, wherein the compound represented by Formula 1 is any one compound selected from the compounds represented by Formulae 3 to 14:

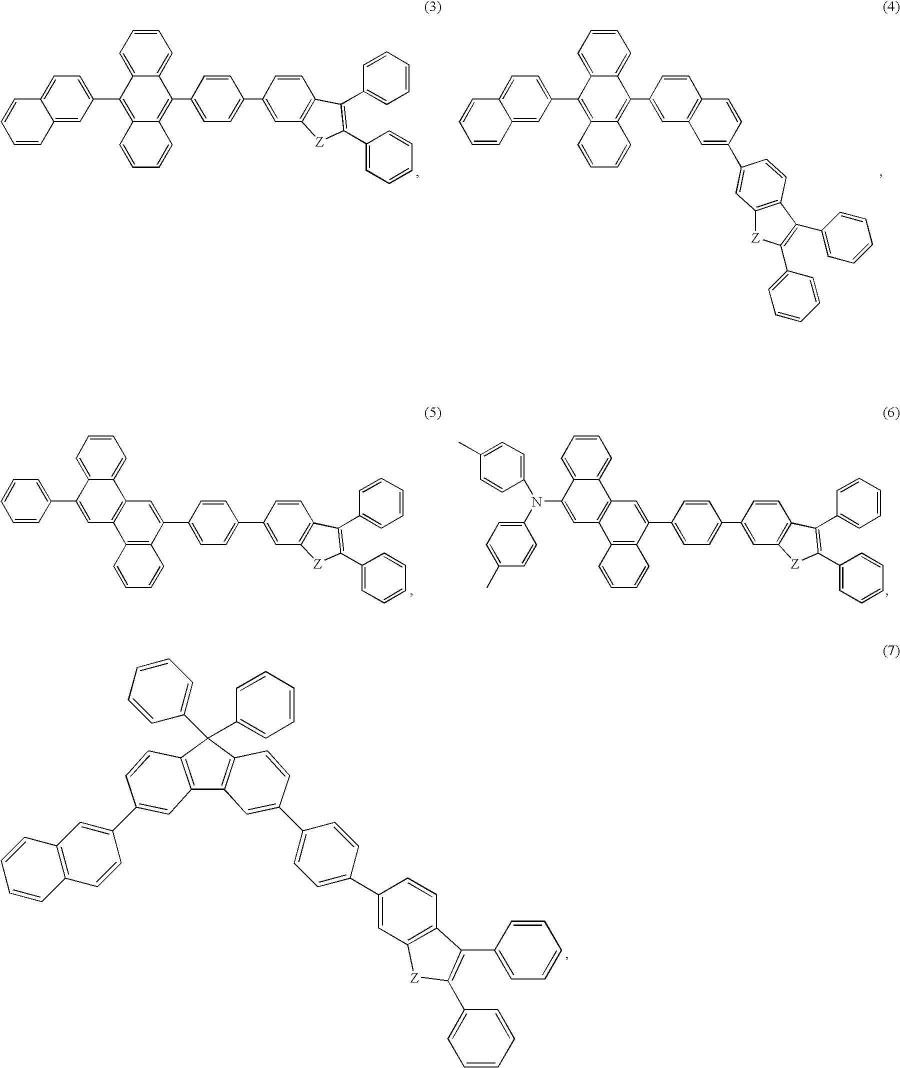

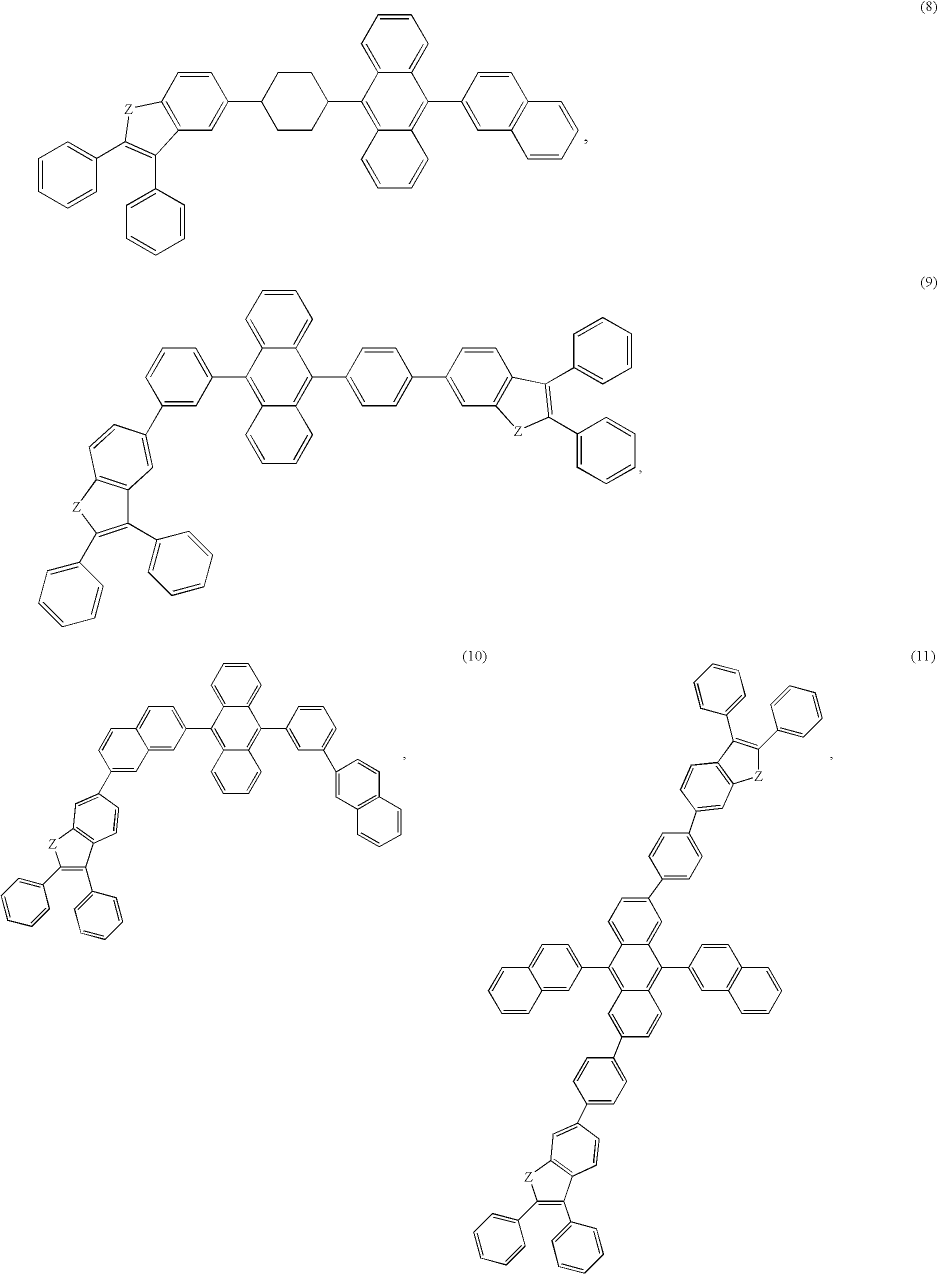

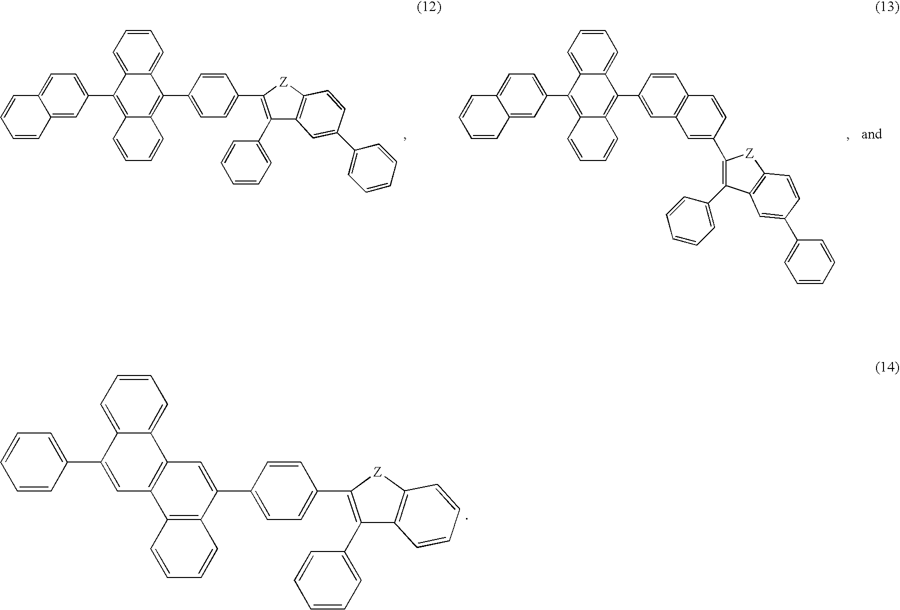

19. The compound of claim 1, wherein the compound represented by Formula 1 is any one compound selected from the compounds represented by Formulae 15 to 23:

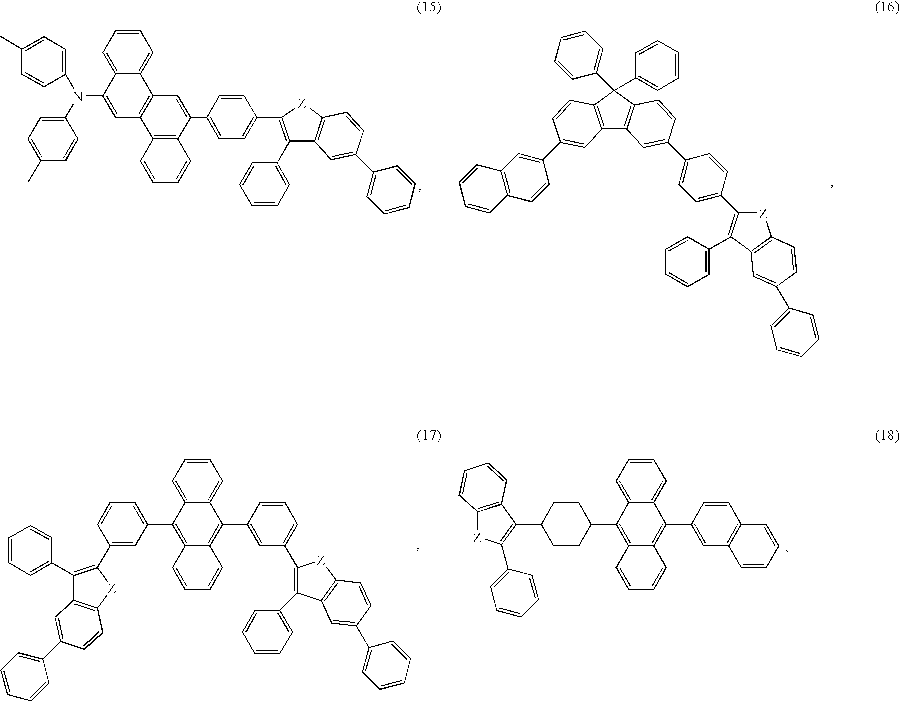

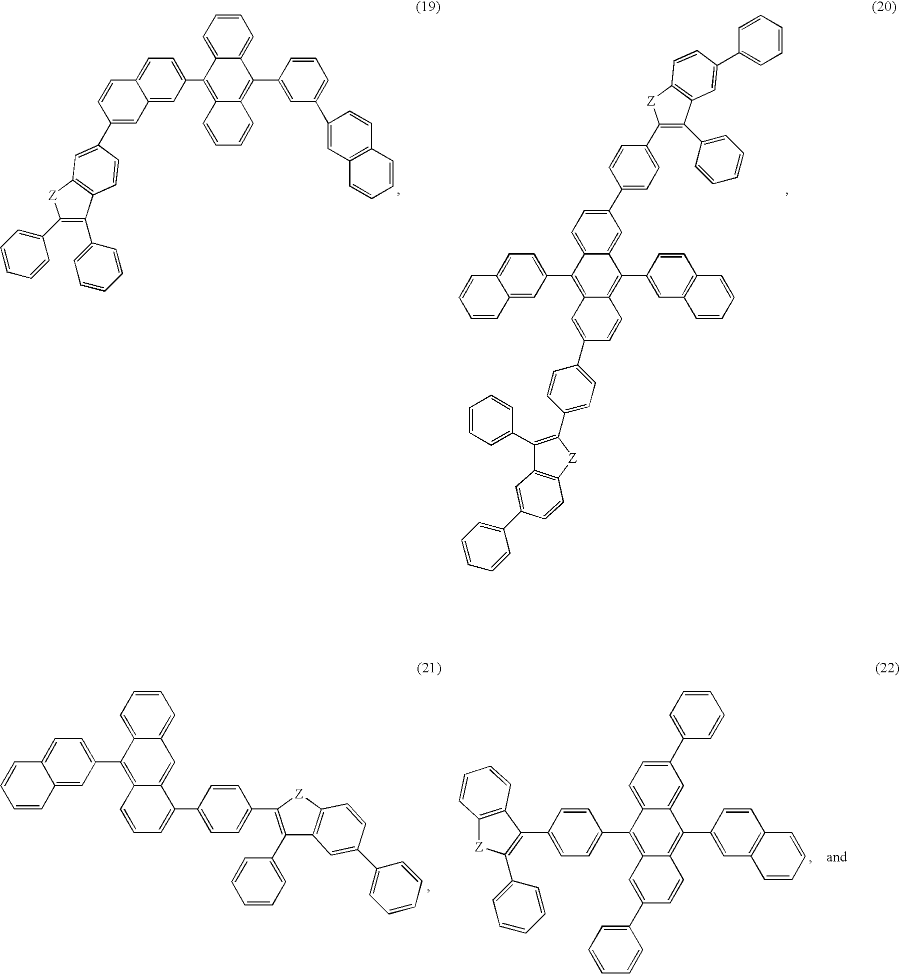

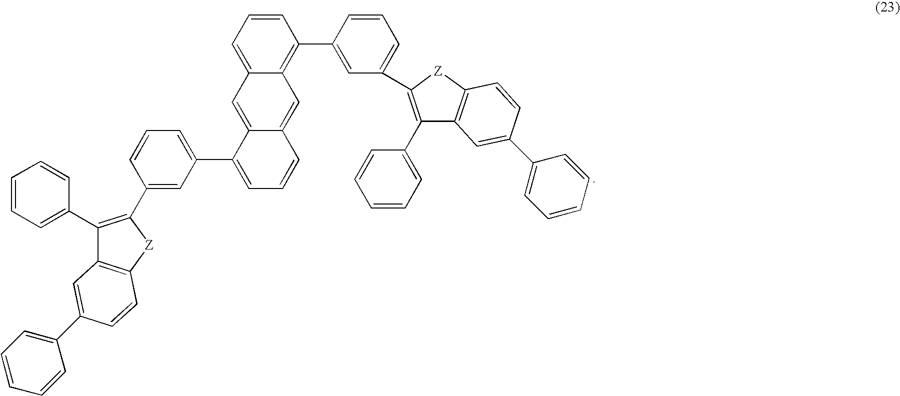

20. An organic light emitting device comprising:
a first electrode;
a second electrode; and
at least one organic layer interposed between the first electrode and the second electrode, said at least one organic layer comprising an organic layer comprised of a compound represented by Formula 1:
X—Ar₁—Ar₂—Y (1)
where Ar₁ is represented by one selected from the groups consisting of the following Formulae: 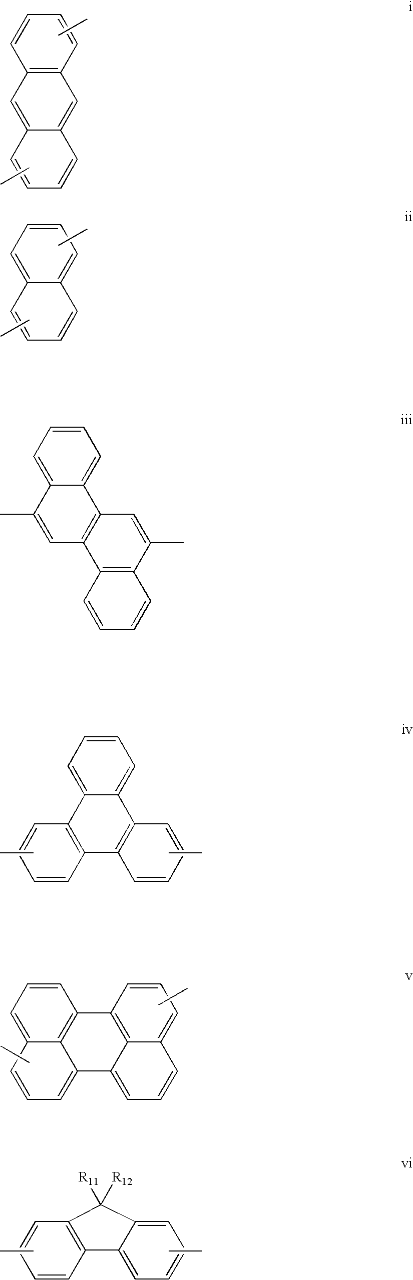 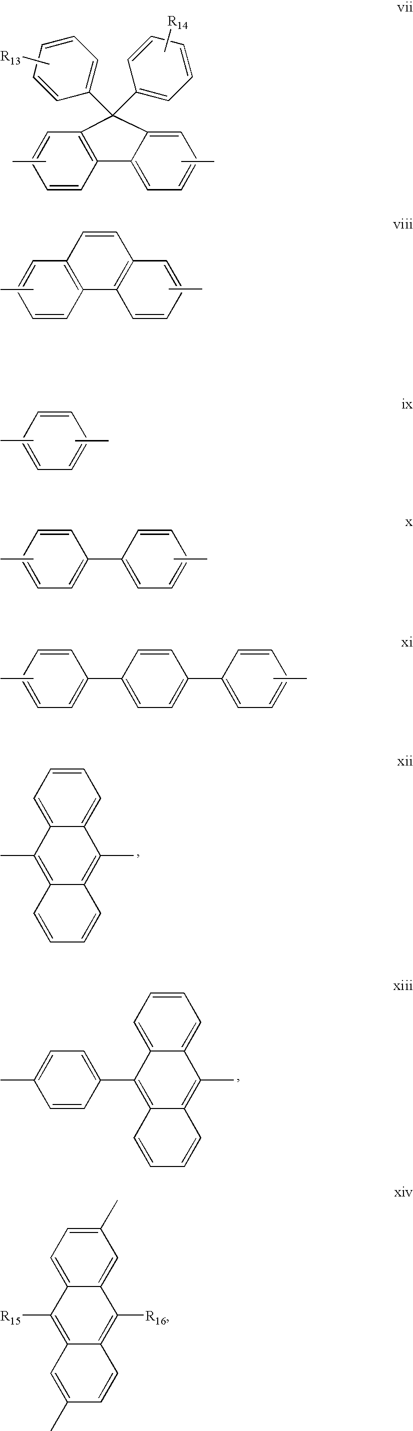 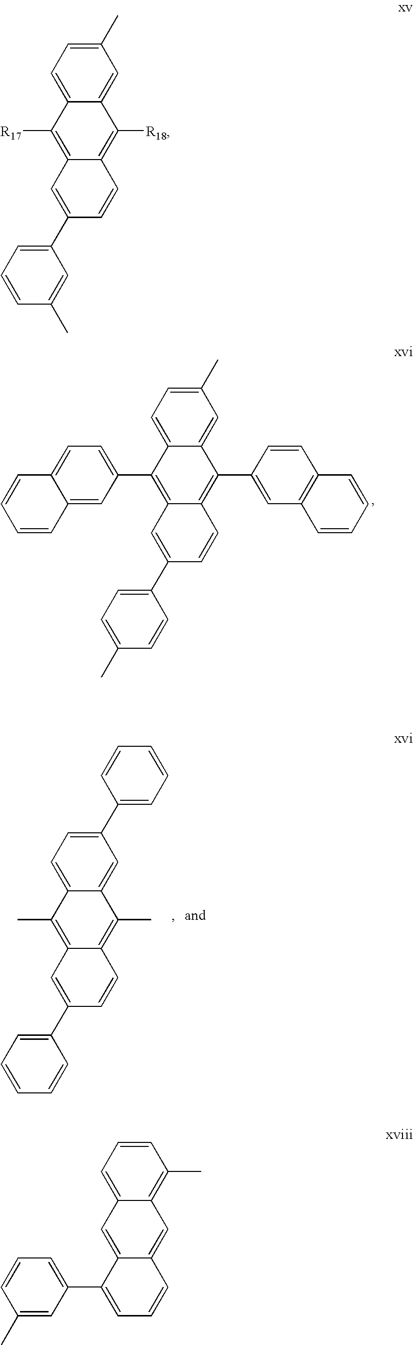
where R₁₁, R₁₂, R₁₃, R₁₄, R₁₅, R₁₆, R₁₇ and R₁₈ are each independently hydrogen, a substituted or unsubstituted C₁-C₂₀ alkyl group, a substituted or unsubstituted C₆-C₃₀ aryl group, a substituted or unsubstituted C₂-C₃₀ heteroaryl group, a substituted or unsubstituted C₅-C₂₀ cycloalkyl group or a substituted or unsubstituted C₅-C₃₀ heterocycloalkyl group;
Ar₂ has one of the following structures: 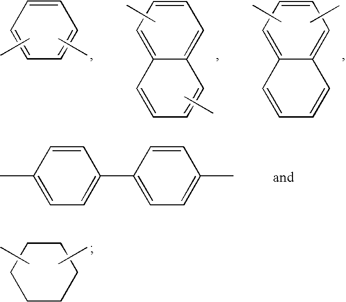
X is each independently hydrogen, halogen, a cyano group, a hydroxyl group, a substituted or unsubstituted C₁-C₂₀ alkyl group, a substituted or unsubstituted C₃-C₂₀ cycloalkyl group, a substituted or unsubstituted C₅-C₂₀ heterocycloalkyl group, a substituted or unsubstituted C₁-C₂₀ alkoxy group, a substituted or unsubstituted C₆-C₃₀ aryl group, a substituted or unsubstituted C₆-C₃₀ arylalkyl group, a substituted or unsubstituted C₂-C₃₀ heteroaryl group, a substituted or unsubstituted C₃-C₃₀ aryl amino group, a substituted or unsubstituted C₃-C₃₀ arylsilane group, a group represented by Formula 2a or a group represented by Formula 2b; and
Y is a group represented by Formula 2a or Formula 2b: 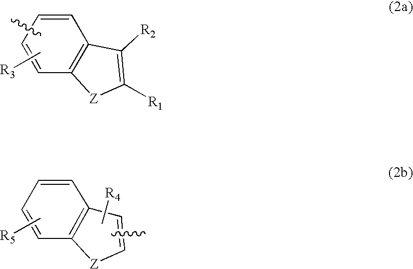
where Z is S, 0, N, Se, or S0₂;
R₁ through R₅ are each independently hydrogen, a C₁-C₂₀ alkyl group, a C₁-C₂₀ alkoxy group, a phenyl group, a biphenyl group, a pentalenyl group, an indenyl group, a naphthyl group, a biphenylenyl group, an anthracenyl group, an azurenyl group, a heptalenyl group, an acenaphthylenyl group, a phenalenyl group, a fluorenyl group, a methylanthryl group, a phenanthrenyl group, a triphenylenyl group, a pyrenyl group, a chrysenyl group, an ethyl-chrysenyl group, a picenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a fluorenyl group, a pyranthrenyl group, an ovalenyl group, a carbazolyl group, a thiophenyl group, an indolyl group, a purinyl group, a benzimidazolyl group, a quinolinyl group, a benzothiophenyl group, a parathiazinyl group, a pyrrolyl group, a pyrazolyl group, an imidazolyl group, an imidazolinyl group, an oxazolyl group, a thiazolyl group, a triazolyl group, a tetrazolyl group, an oxadiazolyl group, a pyridinyl group, a pyridazinyl group, a pyrimidinyl group, a pyrazinyl group, a thianthrenyl group, a cyclopentyl group, a cyclohexyl group, an oxiranyl group, a pyrrolidinyl group, a pyrazolidinyl group, an imidazolyl group, a piperidinyl group, a piperazinyl group, a morpholinyl group, a di(C₆-C₅₀ aryl)amino group, a tri(C₆-C₅₀aryl)silyl group, or derivatives thereof; and [symbol]
denotes a linking group.
